(19) United States
(12) Patent Application Publication
(10) Pub. No.: US 2016/0215344 A1
(43) Pub. Date: Jul. 28, 2016

(54) Methods of Identifying and Treating Subjects having Inflammatory Subphenotypes of Asthma

(71) Applicants: National Jewish Health, Denver, CO (US); The Regents of the University of California, Oakland, CA (US)

(72) Inventors: Max A. Siebold, Denver, CO (US); Esteban Burchard, Danville, CA (US)

(21) Appl. No.: 15/008,094

(22) Filed: Jan. 27, 2016

Related U.S. Application Data
(60) Provisional application No. 62/108,294, filed on Jan. 27, 2015.

(51) Int. Cl. C12Q 1/68

(52) U.S. Cl. CPC C12Q 1/6883; C12Q 1/6881; C12Q 2600/158; C12Q 2600/106

(57) ABSTRACT

The present invention is directed toward novel methods to identify and treat subjects having inflammatory asthma subphenotypes.

### CROSS-REFERENCE TO RELATED APPLICATIONS

This application claims the benefit of priority under 35 U.S.C. 119(e) to U.S. Provisional Patent Application No. 62/108,294, filed Jan. 27, 2015. The disclosure of U.S. Provisional Patent Application No. 61/108,294, is incorporated herein by reference.

### GOVERNMENT SUPPORT

This invention was made with government support under grant number K12HL090147 received from the Natlonal Institutes of Health. The government has certain rights in the invention.

### FIELD OF THE INVENTION

The present invention is directed toward novel methods to identify and treat subjects having inflammatory subphenotypes of asthma.

### BACKGROUND OF THE INVENTION

Asthma is the most common chronic childhood disease, affecting ˜8.7 million children in the United States, and is characterized by chronic inflammation in the airways leading to reversible airway obstruction (Natlonal Health Interview Survey 2004-2011. In: Prevention CfDCa, editor. Natlonal Center for Health Statistics.). Asthma is a disease of the bronchial and lung airways and therefore endotyping and subphenotyping of asthma has focused on the bronchial and lung airways.

Transcriptional profiling of the bronchial airways has shown Th2 inflammation is present in only ˜50% of subjects with asthma revealing the phenotypic heterogeneity of asthma (Woodruff P G, et al. T-helper type 2-driven inflammation defines major subphenotypes of asthma. Am J Respir Crit Care Med 2009; 180: 388-395). Bronchial airway gene expression changes are associated with inhaled corticosteroid response (Woodruff P G, et al. Genome-wide profiling identifies epithelial cell genes associated with asthma and with treatment response to corticosteroids. Proc Natl Acad Sci USA 2007; 104: 15858-15863.), clinical characteristics like eosinophil levels (Bhakta N R, et al. A VCR-based metric of Th2 airway inflammation in asthma. Clin Transl Allergy 2013; 3: 24.), and have identified candidate genes (e.g. CLCA1) (Yurtsever Z, et al. Self-cleavage of human CLCA1 protein by a novel internal metalloprotease domain controls calcium-activated chloride channel activation. J Biol Chem 2012; 287: 42138-42149.) that upon further study have increased understanding of asthma pathogenesis. However, the widespread application of these methods and findings to childhood asthma in large research studies and eventually clinical practice is impeded by the safety, expense, and time limitations presented by obtaining bronchoscopy brushings in children.

Microarray-based expression profiling of bronchial airway epithelium brushings has revealed multiple genes whose expression is dysregulated in adult asthma (Woodruff P G, et al. Genome-wide profiling identifies epithelial cell genes associated with asthma and with treatment response to corticosteroids. Proc Natl Acad Sci USA 2007; 104: 15858-15863.). These studies found a pattern of Th2-driven inflammation that was characterized by expression of calcium-activated chloride channel regulator 1 (CLGA1), periostin (POSTN), and serpin peptidase inhibitor, clade B (SERPINB2) (Woodruff P G, et al. Genome-wide profiling identifies epithelial cell genes associated with asthma and with treatment response to corticosteroids. Proc Natl Acad Sci USA 2007; 104: 15858-15863.; Woodruff P G, et al. T-helper type 2-driven inflammation defines major subphenotypes of asthma. Am J Respir Crit Care Med 2009; 180: 388-395). This so-called “Th2-high” pattern was restricted to a subgroup (˜50%) of the asthmatics screened, reflective of the known phenotypic heterogeneity of asthma (Woodruff P G, et al. T-helper type 2-driven inflammation defines major subphenotypes of asthma. Am J Respir Crit Care Med 2009; 180: 388-395). The Th2-high subphenotype appeared to have clinical significance due to its association with improved inhaled corticosteroid response, higher IgE levels, and higher peripheral blood eosinophils (Woodruff P G, et al. T-helper type 2-driven inflammation defines major subphenotypes of asthma. Am J Respir Crit Care Med 2009; 180: 388-395). Given that there are multiple novel biologic compounds targeting (Corren J, et al. Lebrikizumab treatment in adults with asthma. N Engl J Med 2011; 365: 1088-1098.; Wenzel S, et al. Dupilumab in persistent asthma with elevated eosinophil levels. N Engl J Med 2013; 368: 2455-2466) components of the Th2 inflammatory pathway (Goff L, et al. Visualization and Exploration of Cufflinks High-throughput Sequencing Data. 2012.; Li J and Tibshirani R. Finding consistent patterns: A nonparametric approach for identifying differential expression in RNA-Seq data. Stat Methods Med Res 2011), the ability to profile expression changes in the asthma-affected airway is valuable for not only for elucidating the pathogenesis of asthma but also for predicting and monitoring response to therapy and tailoring individual treatment regimens. However, carrying out bronchoscopy for evaluation of endotype and response to therapy is an invasive process. An alternative to bronchial brushings would increase the practical utility of such findings especially in children.

### SUMMARY OF THE INVENTION

One embodiment of the present invention is a method of identifying a subject at risk of exacerbation of a respiratory disease comprising obtaining a nasal epithelium sample from the subject; determining the expression level of any one or more genes that had been determined to be strongly correlated with IL13 expression from the nasal epithelium sample from the subject; comparing the expression level from the subject to a control level; identifying the subject as being at risk of exacerbation of a respiratory disease if an altered gene expression level of the one or more genes from the subject as compared to the control level is determined.

Another embodiment of the present invention is a method of identifying a subject having a respiratory disease who is responsive to treatment with an inhibitor selected from the group consisting of IL-13, IL-4, IL-5 and Th2 pathway inhibitor comprising obtaining a nasal epithelium sample from the subject; determining the expression level of any one or more genes that had been determined to be correlated with IL-13 expression in the nasal epithelium sample from the subject; comparing the expression level from the subject to a control level; and identifying the subject as being responsive to treatment with the inhibitor if an altered gene expression level of any one or more of the genes from the subject as compared to the control level is determined.

Another embodiment of the present invention is a method of identifying a subject having a Type 2 helper T cell-high (Th2-high) asthma subphenotype comprising obtaining a nasal epithelium sample from the subject; determining the expression level of any one or more genes that had been determined to be strongly correlated with IL-13 expression in the nasal epithelium sample from the subject; comparing the expression level from the subject to a control level; and identifying the subject as having the Th2-high asthma subphenotype if an altered gene expression level of any one or more of the genes from the subject as compared to the control level is determined.

Another embodiment of the present invention is a method to identify a subject having an inflammatory disease resistant to corticosteroid treatment comprising obtaining a nasal epithelium sample from the subject; determining the expression level of any one or more genes that had been determined to be strongly correlated with IL-13 expression in the nasal epithelium sample from the subject; comparing the expression level from the subject to a control level; and identifying the subject as having an inflammatory disease resistant to corticosteroid treatment if an altered gene expression level of any one or more of the genes from the subject as compared to the control level is determined.

In any of the embodiments of the invention described herein, the respiratory disease is asthma.

In any of the embodiments of the invention described herein, the nasal epithelium sample is obtained by a method selected from the group consisting of nasal epithelial brushing or swabbing, nasal lavage, scrapings from a nasal mucosa and blown secretions.

In any of the embodiments of the invention described herein, the expression level of the one or more genes that had been determined to be strongly correlated with IL-13 expression is determined by Next-generation based sequencing and transcript quantification.

In any of the embodiments of the invention described herein, the one or more genes that had been determined to be strongly correlated with IL-13 expression is selected from the group consisting of IL-13, IL-4, IL-5, DPP4, ADRB2, AKAPI2, BCL2A1, C16orf54, C1QA, C1QB, C3, CCL26, CCL5, CD14, CD69, CDH26, CDK14, CLC, CLCA1, CPA3, CSF2RB, CST1, CST4, CXCL9, CXCR1, CXCR2, DHX35, DMXL2, DPYSL3, DUOXA2, EGR1 FFAR2, FFAR3, FHOD3, FOS, G0S2, GPR128, GPR97, GSDMA, GSDMB, HCAR3, HLA-DQA1, IKZF3, IL18R1, IL1B, IL1RL1, IL2RB, IL33, KCNIP4, KLK3, KRT14 KRT16, KRT5, KRT6A, LAG3, LGALS7B, MFGE8, MMP12, MS4A2, MUC21, MUC22, MUC5B, MUC7, MXRA7, NDRG1, NPB, ORMDL3, OSM, P2RY14, POSTN, PRR4 PRSS33, PTHLH, PXDN, PYHIN1, RGS2, SAMSN1, SCGB3A1, SCLY, SCNN1G, SDK2, SEC14L1, SERPINB2, SHISA2, SLC2A3, SLC6A8, SLC7A1, SMAD2, SMAD3, SOCS3, SOX2, SRGN, STEAP4, STOM, TGFB1, THBS1, TLR4, TMEM45A, TPSAB1, TPSB2, TREML2, TSLP, WBSCR17, ZMAT2, and ZPBP2 and combinations thereof.

In any of the embodiments of the invention described herein, the gene expression level of any of the one or more of the genes from the subject as compared to the control level is altered if the expression level of the one or more genes is over-expressed or under-expressed as compared to the control level.

### BRIEF DESCRIPTION OF THE DRAWINGS

FIGS. 1A-1H shows the comparison of non-ubiquitous gene expression between airway tissues. Overlap of expressed genes between nasal-bronchial (FIG. 1A), nasal-small airway epithelium (nasal-SAE) (FIG. 1B), bronchial-SAE (FIG. 1C), and between all tissues (FIG. 1D). Scatter plot of mean expression levels for genes commonly expressed between nasal-bronchial (FIG. 1E), nasal-SAE (FIG. 1F), bronchial-SAE (FIG. 1G). Correspondence-at-top plot for the top 500 genes ranked by expression level from highest to lowest for each tissue (FIG. 1H).

FIG. 2 shows the unsupervised clustering of subjects with atopic asthma and healthy controls using nasal transcriptome expression levels. FPKM (i.e. fragments per kilobase of transcript per million reads) expression levels for all genes in the nasal whole transcriptome sequencing data were used for clustering.

FIG. 3 shows the comparison of gene expression fold-changes in asthma between bronchial and nasal airway expression data for bronchial airway biomarker genes. Scatter plot of previously reported bronchial airway gene expression log 2 fold-changes in asthma, for the top 20 up- and down-regulated genes, versus the fold-changes in asthma for these genes in the nasal airway transcriptome data. Linear regression best-fit line shown.

FIG. 4 shows the correlation between Ampliseq nasal gene expression of IL13 and the other 47 genes differentially expressed in asthma. Genes are ranked from top to bottom by decreasing Spearman correlation coefficient (ρ). Top shaded region and lower shaded region correspond to levels of high positive (ρ>0.5) and negative (ρ<−0.5) correlation, respectively. Significant IL13 correlations=grey bars, Non-significant IL13 correlations=black bars.

FIG. 5 shows the clustering of Ampliseq nasal gene expression levels in study subjects. Clustering was generated using relative nasal expression levels for the 70 genes differentially expressed in atopy (n=99). Heat map represents normalized expression counts (darker shaded regions indicate low; lighter shaded regions indicate high) for each gene. The subject presence (lighter shading in bottom box) or absence (darker shading in bottom box) of atopy, asthma, eosinophil levels, and rhinitis are displayed directly below the heatmap. White squares=missing data.

FIGS. 6A-6C show boxplots of genes differentially expressed in asthma but not atopy the nasal airway. Ampliseq normalized expression counts for 3 of the 6 genes (MUC5B (FIG. 6A), OSM (FIG. 6B), KRT5 (FIG. 6C)) differential expressed in asthma but not atopy (+1 pseudocount and log 10 scale) are plotted according to subject asthma and atopy status.

FIG. 7 shows a representative nasal brush, smear staining. Wright stained nasal brush cell smear, showing nasal airway epithelial cells.

FIG. 8 shows nasal airway epithelium whole transcriptome gene expression in healthy individuals. Expressed genes are categorized by low (0.125-1 FPKM), medium (1-10 FPKM), and high (>10 FPKM). The expression distribution of all genes (top line), and for those where genes ubiquitously expressed across multiple tissues, as described by Ramsköld et al. (An abundance of ubiquitously expressed genes revealed by tissue transcriptome sequence data. PLoS Comput Biol 2009; 5: e1000598), have been removed (bottom line).

FIG. 9 shows a scatter plot of Ampliseq gene expression versus Nasal Whole Transcfiptome (WTS) gene expression data. Data shown for the 20 overlapping subjects and genes between the two expression methods. FPKM cut-off 0.125. Spearman correlation coefficient shown.

FIG. 10 shows IL13 Ampliseq nasal gene expression levels categorized by asthmatic exacerbations. Boxplots display normalized IL13 expression count data for subjects requiring an asthma-related ER visit in the past year (n=30) compared to those that did not (n=19). A one-read pseudocount was added to each sample in order to allow log transformation of genes with expression values of zero. P-value for differential expression between the groups was calculated using a non-parametric Wilcoxon Mann-Whitney test.

FIG. 11 shows the DPP4 gene expression as measured by targeted RNA-seq in 100 Puerto Rican children from the Genes environments and Admixture in Latino Americans (GALA II) study including 50 controls and 50 asthmatics. Controls and asthmatics are stratified by Phadiatop (blood-based) atopy status

FIGS. 12A and 12B show pairs of Air-liquid interface (ALI) differentiated bronchial epithelial cells (BEC) from 5 donors that were treated for 24 hours with IL-13 cytokine (10 ng/ml) and vehicle control. (FIG. 12A) RNA was extracted from all control and IL-13 treated ALI membranes. RNA was used in 5′ nuclease qPCR assays to determine DPP4 and housekeeping gene expression. Normalized DPP4 expression for the control and IL-13 treated cultures of a subject are connected by a line. Well-differential nasal airway epithelial cultures were generated and tested for a single donor denoted by red points. (FIG. 12B) Western blot of cell lysates for DPP4 in control and IL-13 treated BECs from all five donors.

FIGS. 13A and 13B show primary nasal and bronchial basal airway epithelial cells that were transfected with empty plasmid or a plasmid containing CMV-promoter driven DPP4. (FIG. 13A) Overexpression of DPP4 in AECs. (FIG. 13B) Control and DPP4 transfected cells were infected with HRV-16 (10⁴ TOID₅₀/well). RNA was harvested 24 hours later and screened by qPCR for HRV RNA.

FIG. 14 shows the fold change interferon type 1 (first set of bar graphs for each grouping) and 3 (second set of bar graphs for each grouping) gene expression responses in BECs in response DPP4+−HRV-16 infection.

FIG. 15 shows fold change IL8 (first set of bar graohs for each grouping) and CCL26 (second set of bar graphs for each grouping) expression in BECs in response to DPP4+−HRV-16 infection.

FIG. 16 shows fold change of HRV-16 infection (mRNA) in ALI differentiated bronchial epithelial cells (BECs; first set of bar graphs for each grouping) and nasal epithelial cells (NECs; second set of bar graphs for each grouping) in treated with vehicle or IL-13 (10 ng/ml) for final 10 days of differentiation.

FIG. 17A shows Design of DPP4, HRV-16, Alogliptin DPP4 inhibitor study.

FIG. 17B shows fold change in HRV-16 infection (mRNA) in DPP4 transfected and HRV-16 infected cells without and with several concentrations of Alogliptin DPP4 inhibitor. Order of bars starting from the y-axis: Control, 0.1 nM, 1 nM and 10 nM of DPP4 inhibitor.

### DETAILED DESCRIPTION OF THE INVENTION

This invention generally relates to improved methods and kits for identifying and/or treating respiratory diseases in a subject by utilizing nasal epithelium samples from the subject. Further, the inventors have determined that nasal transcriptome can proxy expression changes in the lung airway transcriptome in asthma. Additionally the inventors have determined that nasal transcriptome can distinguish subphenotypes of asthma and are thus predicted to be a predictor of asthma exacerbations. Respiratory diseases such as asthma are diseases of the bronchial and lung airways, thus endotyping and subphenotyping of such diseases have focused on the bronchial and lung airways. However, carrying out bronchoscopy for evaluation of endotype and response to therapy is an invasive process. The inventors of the present invention have found the surprising result that nasal airways are good surrogates for the bronchial and lung airways. Further, the inventors are the first to relate known bronchial asthma biomarkers to the nasal airways, the first to identify Th2 high subtypes of asthma using nasal expression, and the first to show nasal expression is predictive of asthma exacerbation. Because the nasal airways are not directly affected in respiratory diseases such as asthma, the use of nasal airway expression profiles from nasal epithelium samples have not been previously used for endotyping and subphenotyping of respiratory diseases, for treating respiratory diseases or for determining a subject's response to therapy. In addition, the process of obtaining a nasal epithelial sample from a subject, such as by nasal brushing, is a much less invasive procedure compared with obtaining a bronchial epithelial sample via bronchial brushing and therefore increases the practical utility of nasal epithelium samples. Beyond this, the inventors applied a new sensitive targeted RNA-seq approach to endotyping that allows measurement of important RNA molecules that are difficult to measure using other approaches. This technique is referred to as RNA Ampliseq and is based on multiplexed quantitative PCR enrichment of cDNA amplicons, followed by conversion of amplicons to sequence libraries and Next-generation based sequencing of libraries to generate digital count expression data.

The present invention provides for a method of identifying and/or treating a subject at risk of exacerbation of a respiratory disease by determining the expression level of at least one gene associated with the respiratory disease in a nasal epithelium sample from the subject. The present invention also provides for a method of identifying and/or treating a subject having a respiratory disease who is and/or is predictive to be responsive to treatment with an IL-13, IL-4, IL-5 or Th2 pathway inhibitor. In one aspect, a Th2 pathway inhibitor is antibody. In still another aspect the Th2 pathway inhibitor can be lebrikizumab or reslizumab.

The invention also provides for a method of identifying and/or treating a subject having a corticosteroid-resistant inflammatory disease by determining the expression level of Th2-related genes in a nasal epithelium sample from the subject.

The invention also provides for a method of identifying and/or diagnosing and/or treating a subject having an asthma subphenotype by determining the expression level of one or more genes determined to correlate with IL-13 gene expression, including but not limited to IL-13, IL-4, IL-5 and combinations thereof in a nasal epithelium sample from the subject.

Respiratory diseases include but are not limited to asthma, chronic obstructive pulmonary disease (COPD), cystic fibrosis, pulmonary fibrosis, pneumonia, bronchiecatsis, interstitial lung disease, tuberculosis, allergies, lung cancer, emphysema, bronchiolotis, and pneumoconiosis. In one aspect, the respiratory disease is asthma. In one aspect, the asthma is induced by one or more components of the Th2 inflammatory pathway, including but not limited to IL-13, IL-4, IL-5 and combinations thereof. In another aspect, the asthma is unresponsive to corticosteroid treatment and is thus a corticosteroid resistant disease. In still another aspect, the subject has an atopy or allergies which is typically associated with exaggerated or heightened IgE-mediated immune responses

In one aspect of the methods of the invention, a nasal epithelium sample is obtained by one or more of the following methods selected from nasal brushing or swabbing, nasal lavage, scrapings from the nasal mucosa, and blown secretions. In a preferred aspect, the nasal epithelium sample is obtained by nasal brushing or swabbing.

As used herein, the term “expression”, when used in connection with detecting the expression of a gene, can refer to detecting transcription of the gene (i.e., detecting mRNA levels) and/or to detecting translation of the gene (detecting the protein produced). To detect expression of a gene refers to the act of actively determining whether a gene is expressed or not. This can include determining whether the gene expression is upregulated as compared to a control, downregulated as compared to a control, or unchanged as compared to a control or increased or decreased as compared to a reference level. Therefore, the step of detecting expression does not require that expression of the gene actually is upregulated or downregulated or increased or decreased, but rather, can also include detecting that the expression of the gene has not changed (i.e., detecting no expression of the gene or no change in expression of the gene). In addition, the expression level of one or more genes disclosed herein that are strongly correlated with IL-13 can be differentially expressed.

Expression of transcripts and/or proteins is measured by any of a variety of known methods in the art. For RNA expression, methods include but are not limited to: extraction of cellular mRNA and Northern blotting using labeled probes that hybridize to transcripts encoding all or part of the gene; amplification of mRNA using gene-specific primers, polymerase chain reaction (PCR), and reverse transcriptase-polymerase chain reaction (RT-PCR) and/or RNA Ampliseq, followed by quantitative detection of the product by any of a variety of means; multiplexed quantitative PCR enrichment of cDNA amplicons, followed by conversion of amplicons to sequence libraries and Next-generation based sequencing of libraries to generate digital count expression data; extraction of total RNA from the cells, which is then labeled and used to probe cDNAs or oligonucleotides encoding the gene on any of a variety of surfaces; in situ hybridization; and detection of a reporter gene.

Methods to measure protein expression levels generally include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry, as well as assays based on a property of the protein including but not limited to enzymatic activity or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O'Shannessy et al., 1993, Anal. Biochem. 212:457; Schuster et at, 1993, Nature 365:343). Other suitable assays for measuring the binding of one protein to another include, for example, immunoassays such as enzyme linked immunoabsorbent assays (ELISA) and radioimmunoassays (RIA); or determination of binding by monitoring the change in the spectroscopic or optical properties of the proteins through fluorescence, UV absorption, circular dichroism, or nuclear magnetic resonance (NMR).

In one aspect of the methods of the invention, the expression level of at least one gene associated with the respiratory disease in a nasal epithelium sample from the subject and is determined by a method selected from whole or targeted RNA sequencing, Western blotting, immunoassay, flow cytometry.

When comparing the expression level of any one or more genes that had been determined to be strongly correlated with IL-13 expression in the nasal epithelium sample from the subject to a reference level, it is to be understood that the expression level of the any one or more genes is compared with the same gene or genes from the reference or control. For example, if the expression level of IL-13 and IL-4 are both determined or analyzed, then the expression level of IL-13 from the subject would be compared to the expression level of IL-13 from the reference and likewise, the expression level of IL-4 from the subject would be compared to the expression level of IL-4 from the reference. The expression level of any one or more genes as disclosed herein is considered altered if the expression level of the one or more genes as compared to the expression level of the same one or more genes from the reference is increased or decreased (upregulated or downregulated).

In one aspect, the expression levels of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least forty-five, at least fifty, at least fifty-five, at least sixty, at least sixty-five, at least seventy, at least seventy-five, at least eighty, at least eighty-five, at least ninety, or at least ninety-five of the genes are altered (i.e. over-expressed, increased, under-expressed, decreased or be a combination of expression levels) as compared to the corresponding genes in a reference, wherein one or more of the gene expression levels can be increased (or the genes are upregulated) as compared to the reference expression level, while one or more different gene expression levels can be decreased (or the genes are downregulated) as compared to the reference expression level. In one aspect, the gene expression level of the one or more genes is at least about 5%, at least about 10%, at least about 20%, at least about 30%, at least about 40%, at least about 50%, at least about 60%, at least about 70%, at least about 80%, at least about 90%, or 100% different (i.e. increased or decreased) from the expression level of the reference. In still another aspect, the gene expression level of the one or more genes is at least about a 2 fold, at least about a 3 fold, at least about a 4 fold, at least about a 5 fold, at least about a 10-fold, at least about a 20 fold, at least about a 25 fold, at least about a 30 fold, at least about a 40 fold or at least about a 50 fold difference from the expression level of the reference.

As used herein, reference to a reference or control means a subject who is a relevant reference or control to the subject being evaluated by the methods of the present invention. Such a relevant reference or control includes but is not limited to a subject or group of subjects having asthma or a subject or group of subjects that are disease-free (i.e. non-asthmatic) and are non-smoker(s). The control can be matched in one or more characteristics to the subject. More particularly, the control can be matched in one or more of the following characteristics, gender, age, disease state (such Th2-high asthmatic or Th2-low asthmatics). The reference or control expression level used in the comparison of the methods of the present invention can be determined from one or more relevant reference or control subjects.

In still another aspect, the one or more genes that had been determined to be strongly correlated with IL-13 expression are selected from IL-13, IL-4, IL-5, DPP4, ADRB2, AKAP12, BCL2A1, C16orf54, C1QA, C1QB, C3, CCL26, CCL5, CD14, CD69, CDH26, CDK14, CLC, CLCA1, CPA3, CSF2RB, CST1, CST4, CXCL9, CXCR1, CXCR2, DHX35, DMXL2, DPYSL3, DUOXA2, EGR1, FFAR2, FFAR3, FHOD3, FOS, G0S2, GPR128, GPR97, GSDMA, GSDMB, HCAR3, HLA-DQA1, IKZF3, IL18R1, IL1B, IL1RL1, IL2RB, IL33, KCNIP4, KLK3, KRT14, KRT16, KRT5, KRT6A, LAG3, LGALS7B, MFGE8, MMP12, MS4A2, MUC21, MUC22, MUC5B, MUC7, MXRA7, NDRG1, NPB, ORMDL3, OSM, P2RY14, POSTN, PRR4, PRSS33, PTHLH, PXDN, PYHIN1, RGS2, SAMSN1, SCGB3A1, SCLY, SCNN1G, SDK2, SEC14L1, SERPINB2, SHISA2, SLC2A3, SLC6A8, SLC7A1, SMAD2, SMAD3, SOCS3, SOX2, SRGN, STEAP4, STOM, TGFB1, THBS1, TLR4, TMEM45A, TPSAB1, TPSB2, TREML2, WBSCR17, ZMAT2, and ZPBP2 and combinations thereof. In preferred embodiments, the one or more genes are selected from a group of any 5 or more such genes, 10 or more such genes, 20 or more such genes, 30 or more such genes, 40 or more such genes, 50 or more such genes, 60 or more such genes, 70 or more such genes, 80 or more such genes, 90 or more such genes, or 100 or more such genes. In a preferred aspect, the one or more genes is selected from IL-13, IL-4 and IL-5.

Although nasal brushing samples from the subject are composed primarily (>95%) of airway epithelial cells, there are immune cells present that greatly influence the expression pattern of the nasal epithelium including by production of IL-13. Due to the scarcity of these immune cells previous investigators have been unable to detect IL-13 in the airway. The inventors have used ultra-sensitive targeted NEXT-Generation sequencing technology to measure the level of the potent IL-13 cytokine in the nasal airway samples. Thus in one aspect of the invention, one or more genes associated with the respiratory disease are genes that strongly correlate with IL-13 gene expression. As used herein, the term “strongly correlate” or “strongly correlated” with IL-13 gene expression is defined as a spearman correlation coefficient between with the gene in question and IL-13 levels are either >0.5 (strongly positively correlated) or <−0.5 (strongly negatively correlated).

The methods of the present invention can be used for separating Th2-high from Th2-low asthmatics or subphenotyping (endotyping) the asthmatics. In some cases Th2-low asthmatics have no or very low IL-13 expression whereas Th2-high asthmatics have measurable or high IL-13 expression levels.

The invention also provides for a kit for diagnosing and/or treating an asthma subphenotype. In some aspect, the kits can include labeling probes, gene specific primers, sequencing primers, an antibody, detection ability, and quantification ability. Similar to the bronchial airway epithelium, the nasal airway epithelium is populated by basal, ciliated, and secretory epithelial cells (Harkema J R, et al. The nose revisited: a brief review of the comparative structure, function, and toxicologic pathology of the nasal epithelium. Toxicol Pathol 2006; 34: 252-269.). As such the nasal airway presents an accessible alternative to the bronchial airway that may reflect much of the dysfunction present in the asthmatic bronchial airway. Supporting this, analysis of expression for ˜2300 genes in nasal and bronchial airway brushings indicate a close relationship between these two airway sites (Sridhar S, et al. Smoking-induced gene expression changes in the bronchial airway are reflected in nasal and buccal epithelium. BMC Genomics 2008; 9: 259.). Furthermore, a small study indicated gene expression profiles were altered in the nasal brushings of subjects with asthma versus healthy controls (Guajardo J R, et al. Altered gene expression profiles in nasal respiratory epithelium reflect stable versus acute childhood asthma. J Allergy Clin Immunol 2005; 115: 243-251). Finally, children experiencing asthma exacerbations exhibited altered gene expression in the nasal airway compared to children whose asthma was stable (Guajardo J R, et al. Altered gene expression profiles in nasal respiratory epithelium reflect stable versus acute childhood asthma. J Allergy Clin Immunol 2005; 115: 243-251).

As disclosed herein the inventors used high-depth whole transcriptome sequencing to comprehensively determine the degree to which the nasal airway serves as a biologic proxy for the bronchial airway. The inventors also used novel targeted RNA-sequencing technology to profile gene expression of airway biomarkers in a large group of well-characterized children with asthma and healthy controls. These data were used to determine the relationship between the nasal transcriptome and subphenotypes of asthma.

Microarray-based expression profiling of bronchial airway epithelium brushings has revealed multiple genes whose expression is dysregulated in adult asthma (Woodruff P G, et al. Genome-wide profiling identifies epithelial cell genes associated with asthma and with treatment response to corticosteroids. Proc Natl Acad Sci USA 2007; 104: 15858-15863.). These studies found a pattern of Th2-driven inflammation that was characterized by expression of calcium-activated chloride channel regulator 1 (CLCA1), periostin (POSTN), and serpin peptidase inhibitor, Glade B (SERPINB2) (Woodruff P G, et al. Genome-wide profiling identifies epithelial cell genes associated with asthma and with treatment response to corticosteroids. Proc Natl Acad Sci USA 2007; 104: 15858-15863.; Woodruff P G, et al. T-helper type 2-driven inflammation defines major subphenotypes of asthma. Am J Respir Crit Care Med 2009; 180: 388-395). This so-called “Th2-high” pattern was restricted to a subgroup (˜50%) of the asthmatics screened, reflective of the known phenotypic heterogeneity of asthma (Woodruff P G, et al. T-helper type 2-driven inflammation defines major subphenotypes of asthma. Am J Respir Crit Care Med 2009; 180: 388-395.). The Th2-high subphenotype appeared to have clinical significance due to its association with improved inhaled corticosteroid response, higher IgE levels, and higher peripheral blood eosinophils (Woodruff P G, et al. T-helper type 2-driven inflammation defines major subphenotypes of asthma. Am J Respir Crit Care Med 2009; 180: 388-395.). As there are multiple novel biologic compounds targeting (Corren J, et al. Lebrikizumab treatment in adults with asthma. N Engl J Med 2011; 365: 1088-1098.; Wenzel S. et al. Dupilumab in persistent asthma with elevated eosinophil levels. N Engl J Med 2013; 368: 2455-2466.) components of the Th2 inflammatory pathway (Goff L, et al: Visualization and Exploration of Cufflinks High-throughput Sequencing Data. 2012.; Li J and Tibshirani R. Finding consistent patterns: A nonparametric approach for identifying differential expression in RNA-Seq data. Stat Methods Med Res 2011.), the ability to profile expression changes in the asthma-affected airway is valuable not only in elucidating the pathogenesis of asthma but also for predicting and monitoring response to therapy and tailoring individual treatment regimens.

The inventors have determined that over 90% of non-ubiquitous transcripts expressed in the bronchial airway are also expressed in the nasal airway, with strong correlation between expression of the two transcriptomes. The correlation in gene expression between the nasal and lung small airways was also strong, indicating that the nasal airways can also serve as a good surrogate for the bronchial and small airways. The results presented herein confirm and now extend to a whole transcriptome level the previous observations, which reported large similarities in bronchial and nasal expression of surface receptor genes (McDougall C M, et al. Nasal epithelial cells as surrogates for bronchial epithelial cells in airway inflammation studies. Am J Respir Cell Mol Biol 2008; 39: 560-568.), and a subset of bronchial genes examined by microarray (Sridhar S, et al. Smoking-induced gene expression changes in the bronchial airway are reflected in nasal and buccal epithelium. BMC Genomics 2008; 9: 259). Strikingly, the inventors found unsupervised clustering of the nasal whole transcriptome data resulted in near complete separation of atopic asthmatics from healthy controls.

The inventors also demonstrate an IL13 centric Th2 inflammation in the nasal airways of subjects with asthma that is analogous to the Th2 inflammation previously observed in the bronchial airways. The largest study of differential gene expression in the asthmatic bronchial airways identified a clear pattern of Th2-driven inflammation marked by the IL-13 responsive genes POSTN, CLCA1, and SERPINB2. The inventors found both IL13 and these IL-13 responsive genes were all differentially expressed among subjects with asthma in their large targeted RNA-seq screen of the nasal airways. Moreover, 68% of the genes the inventors associated with asthma in the targeted RNA-seq screen were strongly correlated with IL13 levels. Additionally, the inventors found the direction and fold-change of the top 40 differentially expressed genes in the bronchial airways was mirrored by the nasal transcriptome differential expression results.

As expected nasal Th2 inflammation was associated with rhinitis, but 17 Th2-high subjects had atopy but not rhinitis, showing the nasal signature is not just a manifestation of rhinitis, but rather a risk factor for rhinitis. The common pattern of Th2 inflammation in both the bronchial and nasal airways is believed to be driven by an underlying Th2 skew systemic immune system. Supporting this, both atopy status and blood eosinophils levels were strongly associated with the Th2-high pattern of nasal gene expression, regardless of asthma status. Moreover, the Th2-high nasal expression pattern was present in 64% of subjects with asthma and the inventors found 29 of these 32 subjects were atopic. Likewise, the bronchial airway profiling study found a Th2-high expression pattern in 53% of subjects with asthma, which were characterized by higher IgE levels and both higher blood and bronchoalveolar lavage eosinophils. These results support that Th2 airway inflammation is a part of the mechanistic basis of asthma in atopic or more systemically allergic individuals.

The inventors have found that the identification of Th2-high subjects is feasible by nasal brushings. The ability to stratify subjects with asthma by the presence of Th2 airway inflammation creates a more homogeneous group of subjects with regard to asthma pathogenesis and increase the power of future biomedical and clinical research studies. Moreover, this stratification allows the search for the genetic determinants of non-Th2 driven asthma, for which little is known regarding disease pathogenesis. The fact that 9 of the 16 asthma GWAS (genome-wide association study, also referred to as whole genome association study) genes tested were differentially expressed in the nasal epithelium suggest there may be a genetic basis to these disease subphenotypes. The potential clinical utility of the bronchial Th2 inflammation signature has been shown, as response to inhaled steroids was almost entirely restricted to the bronchial Th2-high subjects. Moreover, a Th2-high airway status would be a highly relevant biomarker in Th2 targeted therapeutic trials. Supporting this, serum levels of periostin have recently been used as biomarker in clinical trial of an IL-13 inhibitor (Corren J, et al. Lebrikizumab treatment in adults with asthma. N Engl J Med 2011; 365: 1088-1098.). Finally, the inventors association of nasal IL-13 levels with asthma exacerbations suggests nasal airway expression levels may be predictive of loss in asthma control and useful for clinical management.

Surprisingly, the inventors have identified six genes that were differentially expressed in asthma and not atopy. This observation suggests that there exists dysregulation in nasal airway expression beyond Th2 inflammation that is relevant to both atopic and non-atopic asthma. Nasal expression of both KRT5, a marker of basal airway epithelial cells, and MUC5B, a marker of secretory cells, was downregulated in asthma. Changes in the expression of these genes may reflect airway remodeling of subjects with chronic asthma, characterized by changes in the cellular and mucosal composition of the airway. Nasal overexpression of the OSM gene supports this idea, as OSM gene and protein expression (an interleukin-6 family member) has previously been shown to be upregulated in the sputum of asthmatics with irreversible airway obstruction (Simpson J L, et al. Oncostatin M (OSM) is increased in asthma with incompletely reversible airflow obstruction. Exp Lung Res 2009; 35: 781-794).

The inventors have also determined that DPP4 is highly upregulated in asthmatics and in particular atopic asthmatics. DPP4 is a protease that has been implicated in inflammatory responses. Recent studies of a DPP4 knockout rat model reveal deficiency of DPP4 results in protective effects on airway inflammation in experimental asthma. DPP4 has been implicated in binding of the human immunodeficiency virus to T-cells and a Coronavirus to airway epithelial cells. Based on these observations DPP4 expression in the airway epithelium is expected to be inducible by the Th2 cytokine, IL-13, and further DPP4 may contribute to asthma by modulating anti-viral responses of the airway epithelium. Based on the findings by the inventors, the expression of DPP4 alone or in combination with other genes of the Th2 pathway, may predict a subject's response to inhaled corticosteroids, predict a subject's risk for asthma exacerbation as well as predicting or allowing for the monitoring of a subject's response to Th2 blockers.

The inventors have determined that the nasal airways are an excellent less-invasive proxy for the bronchial airways in transcriptional profiling studies. The pattern of Th2 inflammation in nasal airways is highly similar to the Th2 pattern observed in the bronchial airways of subjects with asthma. This Th2 airway inflammation signature is highly enriched in atopic asthmatics. These data clearly show the usefulness of nasal airway brushings for subphenotyping of children with asthma in both research and clinical settings.

The following examples are provided for illustrative purposes, and are not intended to limit the scope of the invention as claimed herein. Any variations which occur to the skilled artisan are intended to fall within the scope of the present invention. All references cited in the present application are incorporated by reference herein to the extent that there is no inconsistency with the present disclosure.

### EXAMPLES

### Materials and Methods for Examples 1-5

### Subject Recruitment

Study subjects are a randomly selected subset of Puerto Rico islanders that were recruited as part of the ongoing Genes environments & Admixture in Latino Americans (GALA II) study described elsewhere (Borrell L N, et al. Childhood obesity and asthma control in the GALA II and SAGE II studies. Am J Respir Crit Care Med 2013; 187: 697-702.; Kumar R, et al. Factors associated with degree of atopy in Latino children in a nationwide pediatric sample: The Genes-environments and Admixture in Latino Asthmatics (GALA 11) study. J Allergy Clin Immunol 2013.; Nishimura K K, et al. Early Life Air Pollution and Asthma Risk in Minority Children: The GALA II & SAGE II Studies. Am J Respir Crit Care Med 2013.). Of the 100 study subjects who participated in this study 92 were re-contacted from their original GALA 11 study visit. Asthma was defined by physician diagnosis and the presence of two or more symptoms of coughing, wheezing, or shortness of breath in the 2 years prior to enrollment. Asthma exacerbations were defined by self-report of a subject having asthma symptoms requiring an emergency room visit. Atopy was defined by plasma testing with the Phadiatop Inhalant Multi-allergen Immunocap (Szefler S J, et al. Asthma outcomes: biomarkers. J Allergy Clin Immunol 2012; 129: S9-23.) as a qualitative outcome. All study subjects had no history of smoking or recent nasal steroid use (within 4 weeks of recruitment). The study was approved by local institutional review boards, and written assent/consent was received from all subjects and their parents.

### Nasal Brushing Collection and RNA Extraction

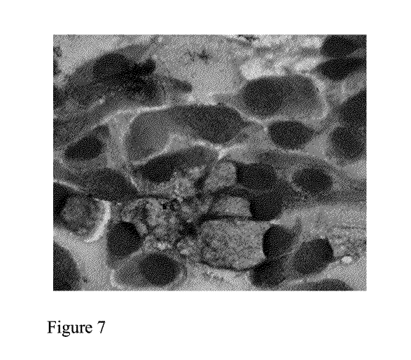

Nasal epithelial cells were collected from behind the inferior turbinate with a sterile cytology brush (Cyto-Soft Cytology Brush CYB-1, Cardinal Health #S7766-1A) using a nasal illuminator. The collected brush was submerged in RLT Plus lysis buffer plus beta-mercaptoethanol and frozen at −80 C until extraction. For some subjects (n=20) the nasal brush was lightly touched to a glass slide to transfer some cells for staining before submerging the brush in lysis buffer. Wright's staining of the slides revealed sheets of airway epithelial cells on all 20 slides (FIG. 7). RNA quality was determined by Agilent Bioanalyzer for 50 of the 100 samples and these samples had a RNA integrity number >8.2.

### Whole Transcriptome Gene Expression

Barcoded Illumina RNA-seq libraries were prepared for each individual using the Illumina Tru Seq RNA Sample Preparation Kit (v2). Libraries were pooled and sequenced across two full flow cells of an Illumina HiSeq 2000. Raw sequencing quality was assessed using the fastx software package (Hannon G J. FASTX-Toolkit. www.hannonlabcshledu/fastx_toolkit/indexhtml.). Reads from each sample were consolidated and mapped to the hg19 genome using Tophat v2.0.6 (Kim D, et al. TopHat2: accurate alignment of transcriptomes in the presence of insertions, deletions and gene fusions. Genome Biol 2013; 14: R36.; Woodruff P G, et al. T-helper type 2-driven inflammation defines major subphenotypes of asthma. Am J Respir Crit Care Med 2009; 180: 388-395.) with Bowtie version 2.1.0 (Langmead B and Salzberg S L. Fast gapped-read alignment with Bowtie 2. Nat Methods 2012; 9: 357-359.). After mapping, fragments were further filtered to remove sequences where mates were unmapped, mapped to another chromosome, or either mate had an alignment quality score <20. Genes and transcripts were identified from mapped reads and quantified using Cufflinks version 2.0.2 (Trapnell C, et al. Transcript assembly and quantification by RNA-Seq reveals unannotated transcripts and isoform switching during cell differentiation. Nat Biotechnol 2010; 28: 511-515.). Transcript assembly was restricted to annotated isoforms using the “−G” option. Differential expression between asthmatics and controls was performed using the Cuffdiff tool (Trapnell C, et al. Differential gene and transcript expression analysis of RNA-seq experiments with TopHat and Cufflinks. Nat Protoc 2012; 7: 562-578.) in the Cufflinks package with default settings. The iGenornes transcript annotation file (UCSC hg19) was downloaded (www.tophat.cbcb.umd.edu/igenomes.shtml) and used in both mapping and transcript assembly, as instructed by the Tophat/Cufflinks documentation.

### Tissue Comparison

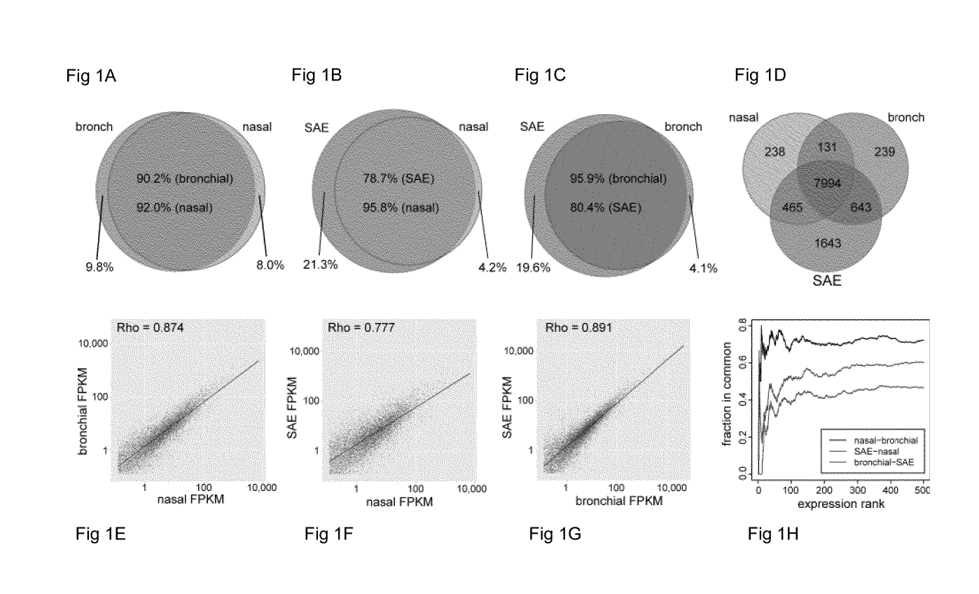

Public RNA-seq data sets were downloaded from the sequence read archive (SRA) (Wheeler D L, et al. Database resources of the Natlonal Center for Biotechnology Information. Nucleic Acids Res 2008; 36: D13-21.). Specifically, bronchial epithelial RNA-seq data was obtained from bronchial brushings of a pooled sample of 3 healthy (race not reported), non-smoking individuals (SRR192333) (Beane J, et al. Characterizing the impact of smoking and lung cancer on the airway transcriptome using RNA-Seq. Cancer Prev Res (Phila) 2011; 4: 803-817.). Small airway epithelium data came from five Black non-smokers (SRR094862, SRR094863, SRR094864, SRR094906, SRR094907) in a study by Hackett, et al (RNA-Seq quantification of the human small airway epithelium transcriptome. BMC Genomics 2012; 13: 82.). For each tissue FPKM expression levels from healthy individuals were averaged across all genes. Genes obtaining a FPKM value of 0.125 or greater were considered expressed for all three data sets, as we considered this biologically significant expression since an FPKM of 0.125 corresponds to ˜1 transcript expressed per 8 cells (Hackett N R, et at RNA-Seq quantification of the human small airway epithelium transcriptome. BMC Genomics 2012; 13; 82.) (FIG. 1). Genes ubiquitously expressed across multiple tissues, as described by Ramsköld, et al (Ramsköld D, et al. An abundance of ubiquitously expressed genes revealed by tissue transcriptome sequence data. PLoS Comput Biol 2009; 5: e1000598.), were removed. Bronchial airway fold-change gene expression changes in asthma were obtained from the Woodruff et al (Genome-wide profiling identifies epithelial cell genes associated with asthma and with treatment response to corticosteroids. Proc Natl Acad Sci USA 2007; 104: 15858-15863) bronchial brushing microarray study at the following website woodrufflab.ucsf.edu/genomics_pub/EPITHtop1000.html.

### Ampliseq Expression Analysis

Selection criteria for a subset of 29 “asthma candidate genes” screened in the Ampliseq analysis were as follows: 23 asthma genetic candidates were selected from GWAS studies (16 genes) and other smaller genetic studies of asthma or related traits (7 genes). An additional 7 biologic candidates implicated in mechanistic studies of asthma were selected. (Note: one gene was selected as both a genetic and biologic candidate, resulting in 29 total genes). Table IV provides a reference for each candidate gene selected. Targeted RNA-seq expression analysis was performed by Ion RNA Ampliseq (Life Technologies). RNA Ampliseq libraries were designed and generated using Life Technologies design software, standard protocol, and reagents from the Ion Ampliseq RNA Library Kit (Life Technologies). Namely, Life Technologies multiplexing software was used to design primers to amplify ˜100 bp amplicons for 105 genes in a multiplex reaction. According to standard library generation protocol: (1) 10 ng of RNA was reverse transcribed, (2) followed by multiplex PCR amplification of cDNA using the multiplex primer panel, (3) adapter and barcode ligation to generate sequencing library, (4) library amplification. RNA Ampliseq libraries were sequenced using the Ion Torrent Proton on three P1 chips (Life Technologies). Reads mapping to target transcript amplicons were tabulated as expression count data using the torrent mapping alignment program (TMAP) and a Life Technologies in-house pipeline. Raw Ampliseq expression values for each gene were determined by counting the number of reads mapping to the corresponding amplicon target. Differential expression analysis was performed on raw count data using the non-parametric SAMseq (Li J, Tibshirani R. Finding consistent patterns: A nonparametric approach for identifying differential expression in RNA-Seq data. Stat Methods Med Res 2011.) method available in the samR package using default settings, except for increasing the number permutations to 10,000. Differential expression testing in the Sam-seq package uses a modified Wilcoxon Statistic to test for differential expression with a multiple resampling strategy to correct for differences in total read counts between samples. Significance of results was determined by generating a null distribution of the modified Wilcoxon statistic by permutation. Significant genes after correcting for multiple testing are determined by False Discovery Rate Method directly computing the q value. The q value represents the expected proportion of false-positives incurred when calling that gene significant. Genes with q-values less than 0.05 were considered to be differentially expressed.

### Statistical Methods

The correlation between gene expression of commonly expressed non-ubiquitous genes from different airway sites was calculated using Spearman's rank correlation coefficient (rho) (FIG. 1). Venn diagrams were produced using the VennDiagram package (v1.5.1) in R. All plots and calculations were performed using the R statistical package and correspondence-at-top plot values were calculated as previously described (Irizarry R A, et al. Multiple-laboratory comparison of microarray platforms. Nat Methods 2005; 2: 345-350.).

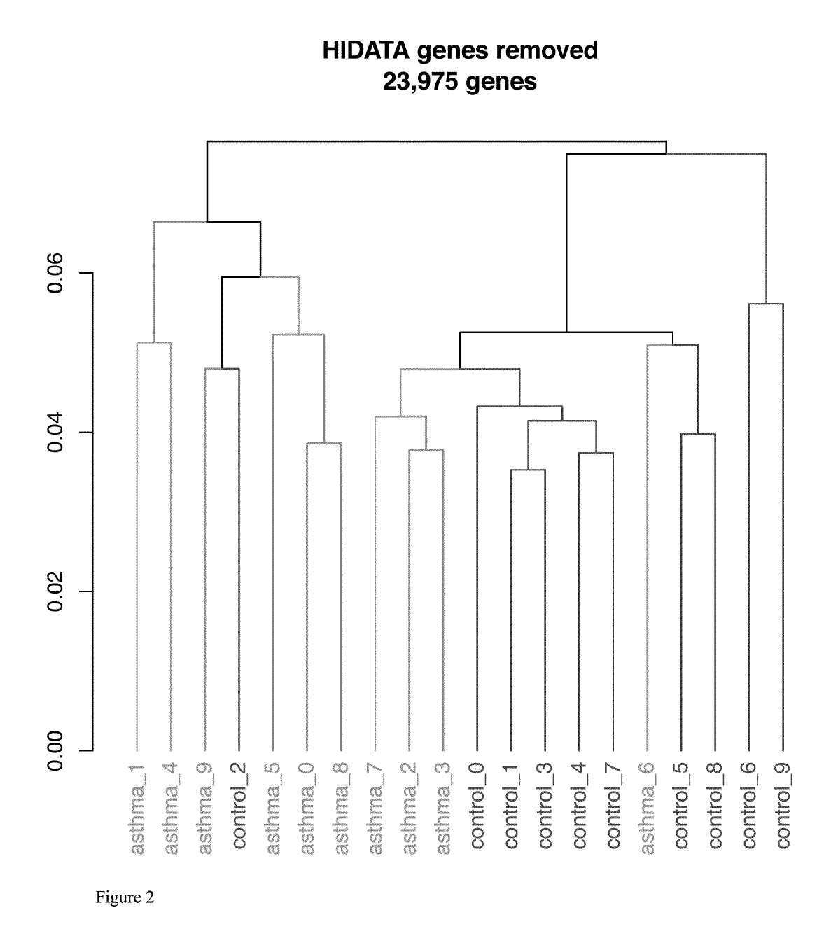

Hierarchical clustering of samples in FIG. 2 was done using the csDendro function in the CummeRbund package (Goff L T, et al: Analysis, exploration, manipulation, and visualization of Cufflinks high-throughput sequencing data. wwwbioconductororg/packages/release/bioc/html/cummeRbundhtml 2012) with default parameters. The method uses the Jensen-Shannon distance and complete linkage for dendrogram construction.

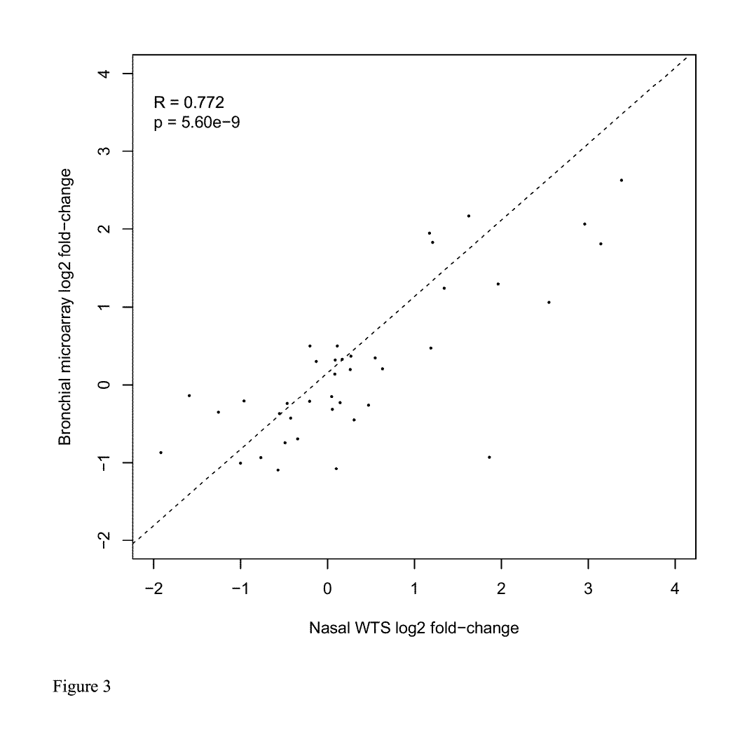

The correlation between log 2 transformed nasal and bronchial asthma fold-changes in gene expression was determined by Pearson correlation coefficient (FIG. 3).

Raw count values were normalized to correct for unequal sample mixing using DESeq (Anders S, and Huber W. Differential expression analysis for sequence count data. Genome Biol 2010; 11: R106.) for display of expression data and clustering analyses. Hierarchical clustering of genes (FIG. 5) used Spearman rank correlation coefficient ρ (as dissimilarity metric [1−ρ]) and complete linkage. Heatmap of expression values (FIG. 5) was generated by transforming normalized counts for each gene into Z-scores, binning, and assigning bin colors from red to green representing low, and high expression levels, respectively, with black representing the mean expression level. Samples were clustered using the proximity score from a Random Forest classifier (Breiman L. Random Forests. Machine Learning 2001: 5-32) trained to segregate samples into atopic or non-atopic status (positive or negative Phadiatop test) using the randornForest R package (Liaw A W, Classification and Regression by randomForest. R News 2002: 18-22.) version 4.6-7 implementation. Random Forest classifier was run using the default settings for number of trees grown (500) and gene sampling size (square root of the total number of genes). Subjects were sampled using an equal number of atopic and non-atopic (20 of each) to construct each tree. The subject sampling strategy was determined empirically by maximizing the classifier accuracy on samples withheld from the training set. To form clusters, the distance between any two samples was equal to one minus the proximity score and clusters were aggregated using complete linkage. Fishers exact test was used to calculate odds ratio P-values for association of clinical phenotypes with Th2-high and -low sample clusters.

### Example 1

This example demonstrates that whole transcriptome gene expression signatures of the nasal airway epithelium mirror the bronchial airway epithelium.

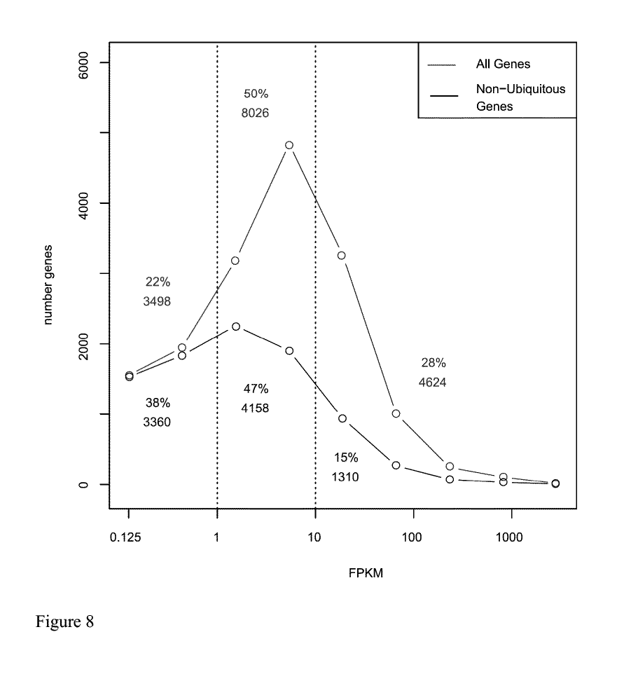

Whole transcriptome sequencing of nasal airway epithelium brushings from 10 non-atopic controls and 10 atopic asthmatics was performed (Table I). Sequencing resulted in an average of 1.1×1.0⁸(+/−4×10⁷) reads mapped per subject (Table II). Mapped reads were used to generate FPKM gene expression levels, which revealed 16,148 expressed genes in the healthy nasal transcriptome (Table II, FIG. 8). Publically available transcriptome sequencing data was accessed to generate transcriptomes for the healthy bronchial and lung small airways (6^{th} generation airways) for comparison with healthy nasal transcriptome (Beane J, et al. Characterizing the impact of smoking and lung cancer on the airway transcriptome using RNA-Seq. Cancer Prev Res (Phila) 2011; 4: 803-817.; Hackett N R, et al. RNA-Seq quantification of the human small airway epithelium transcriptome. BMC Genomics 2012; 13: 82). 7,331 ubiquitously expressed genes were removed from each airway data set, which were defined by expression in a diverse panel of 23 human and murine cell types and tissues (Ramskold D, et al. An abundance of ubiquitously expressed genes revealed by tissue transcriptome sequence data. PLoS Comput Biol 2009; 5: e1000598.). This resulted in 8,828, 9,007, and 10,745 non-ubiquitously expressed genes in the nasal, bronchial, and small airway transcriptomes, respectively. Examining the overlap of the non-ubiquitously expressed nasal and bronchial genes, 90.2% of the genes expressed in the bronchial samples were also found to be present in the nasal airway transcriptome (FIG. 1A). This was only slightly lower than the overlap with small airway epithelium, which expressed 95.9% of the non-ubiquitous bronchial transcriptome (FIG. 1B). In fact, the nasal transcriptome contained 78.7% of the genes expressed in the more distal small airway transcriptome (FIG. 1C, FIG. 1D).

TABLE I
Clinical data for subjects included in the nasal whole transcriptome (WTS) and Ampliseq expression studies

| | WTS | | | Ampliseq | | |
|---|---|---|---|---|---|---|
| | Control | Asthmatic | p-value | Control | Asthmatic | p-value |
| Sample | 10 | 10 | | 50 | 50 | |
| Size | | | | | | |
| Age | 15.2 ± 2.8 | 15.2 ± 2.5 | 0.991 | 15.3 ± 2.8 | 15.0 ± 3.1 | 0.682 |
| Gender | 5/5 | 7/3 | | 22/28 | 30/20 | |
| (M/F) | | | | | | |
| FEV1 %- | 83.4-112.2 (92.1) | 68.8-92.7 (79.0) | 0.0003 | 83.2-126.9 (98.8) | 83.4 ± 16.9 | 2.62e−11 |
| pred | | | | | | |
| Eosinophils * | L: 0.9-8.2 (2.7) | L: 1.3-13.4 (4.7) | L: 0.159 | L: 0.2-11.6 (2.8) | L: 0.7-15.6 (6.2) | L: 0.010 |
| | H: 1.5-8 (5.8) | H: 0.7-0.7 (0.7) | H: 0.500 | H: 0.5-8.6 (2.4) | H: 0.7-14.7 (3.6) | H: 0.226 |
| | | (n = 8) | | | (n = 45) | |
| IgE^{†} | 23.4-219.0 (38.2) | 17.4-2034.5 (158.7) | 0.095 | 4.9-2999.6 (114.6) | 8.6-2992.9 (182.5) | 0.154 |
| | | (n = 9) | | (n = 47) | (n = 45) | |
| Phadiatop (+/−) | 0/10 | 10/0 | | 30/20 | 37/12 | |
| Ancestry ^{‡} | | | | (n = 39) | (n = 45) | |
| African | 0.12-0.51 (0.29) | 0.07-0.19 (0.15) | 0.001 | 0.08-0.51 (0.22) | 0.07-0.49 (0.19) | 0.693 |
| European | 0.43-0.74 (0.59) | 0.66-0.83 (0.73) | 0.001 | 0.43-0.84 (0.68) | 0.43-0.83 (0.69) | 0.534 |
| N. | 0.06-0.14 (0.10) | 0.08-0.16 (0.11) | 0.247 | 0.06-0.22 (0.11) | 0.07-0.25 (0.11) | 0.917 |
| American | | | | | | |

All values are presented as either mean ± SD with P values calculated using a two-sided t-test, or when non-Normal as min-max (median) with P-values calculated by two-sided Wilcoxon Mann-Whitney rank test. Sample sizes differing from “Sample Size” row are noted.
*Data categories are divided into low (L) and high (H) indicating normal ranges of 0.0-4.0 and 0.0-7.0, respectively.

TABLE II
Whole transcriptome sequencing and mapping metrics

| | Control | Asthma | Total |
|---|---|---|---|
| Sample size | 10 | 10 | 20 |
| Read length | 2 × 100 bp | 2 × 100 bp | 2 × 100 bp |
| Avg pairs sequenced | 69,534,854.0 ± 29,853,578 | 54,273,218.5 ± 5,888,143 | 61,904,036 ± 22,358,026 |
| Avg read ends | 139,069,708 ± 59,707,157 | 108,546,437 ± 11,776,285 | 123,808,073 ± 44,716,051 |
| sequenced | | | |
| Reads mapped | 92.56 ± 4.1% | 91.22 ± 4.98% | 91.89 ± 4.49% |
| Mapped reads in genes | 90.49 ± 2.09% | 88.77 ± 2.09% | 89.63 ± 2.22% |
| Mapped reads in exons | 85.25 ± 3.18% | 83.19 ± 2.80% | 84.22 ± 3.10% |
| Mapped reads in | 5.24 ± 1.38 | 5.58 ± 1.00% | 5.41 ± 1.19% |
| introns | | | |
| Mapped intergenic | 9.45 ± 2.10% | 11.17 ± 2.10% | 10.31 ± 2.22% |
| reads | | | |
| Mapped aligning to | 2.77 ± 3.94% | 2.33 ± 1.28% | 2.55 ± 2.86% |
| rRNA | | | |

Values are means and standard deviations calculated on a subject-by-subject basis (not pooled). Reads were mapped with tophat v2.0.6 (using Bowtie2 v2.0.5) and mapping summary statistics were obtained using RNA-SeQC v1.1.7.

Expression levels of these genes were then examined to determine the correlation between the different airway sites. A high correlation (ρ=0.87) was found between the nasal and bronchial transcriptomes, which was similar to the correlation between the bronchial and small airway transcriptomes (ρ=0.89) (FIG. 1E, and FIG. 1G). Strong correlation was also observed between nasal and small airway transcriptomes (ρ=0.78) (FIG. 1F). Additionally, concordance between the three data sets among the 500 most highly expressed genes was examined. 72% overlap of these genes was found between nasal/bronchial airways, 60% between bronchial/small airways, and 47% between nasal/small airways (FIG. 1H). Taken together, these results demonstrate that the composition and structure of the bronchial and lung small airway transcriptome is closely mirrored by that of the nasal airway.

### Example 2

This example demonstrates that the nasal airway transcriptome is altered in atopic asthma and expression changes reflect asthmatic differential expression in the bronchial airway. An unsupervised cluster analysis of the entire nasal transcriptome data set was performed to determine if nasal expression could be used to segregate the 10 atopic asthmatics from the 10 non-atopic healthy controls. Clustering using Jensen-Shannon distance separated asthmatics from controls, with only a few outlier samples in each of two top-level clusters (FIG. 2). An uncorrected analysis of differential expression for all gene transcripts was performed to identify individual gene transcripts that might be driving the separate clustering of asthmatics, for later confirmation in a larger set of subjects. The 50 genes with the largest differential expression statistic are listed in Table III The gene with the lowest p value for differential expression was carboxypeptidase A3 (CPA3), a mast cell gene product. Interestingly, CPA3 was previously identified as the second most differentially expressed gene in the bronchial airway of subjects with asthma (Woodruff P G, et at Genome-wide profiling identifies epithelial cell genes associated with asthma and with treatment response to corticosteroids. Proc Natl Acad Sci USA 2007; 104: 15858-15863.). Therefore, the ability of nasal airway expression to recapitulate the expression pattern in the bronchial airway of subjects with asthma was examined. This was done by comparing the fold changes of the top 20 over- and under-expressed genes in the bronchial airway of subjects with asthma to the fold changes of these genes in the nasal airway of subjects with asthma (Woodruff P G, et at Genome-wide profiling identifies epithelial cell genes associated with asthma and with treatment response to corticosteroids. Proc Natl Acad Sci USA 2007; 104: 15858-15863.

Despite differences in expression platforms (microarray vs. RNA-seq), the direction and magnitude of fold-changes for these 40 bronchial genes were strongly correlated with the asthmatic fold-changes for these genes in our nasal data (ρ=0.77, ρ=5.6×10⁻⁹, FIG. 3). These results support that the nasal airway gene expression profile is altered in asthma and that these changes are reflective of those observed in the bronchial airway of subjects with asthma.

TABLE III
Cufflinks/cuffdiff top 50 differentially expressed genes in nasal whole transcriptome
data

| | | FPKM | FPKM | Fold- | |
|---|---|---|---|---|---|
| Gene | hg19 coordinates | Healthy | Asthmatic | Change | p-value |
| CPA3 | chr3: 148583042-148614872 | 6.25 | 55.65 | 8.897 | 0.003 |
| CLC | chr19: 40221892-40228669 | 0.34 | 27.44 | 80.103 | 0.004 |
| RPPH1 | chr14: 20811229-20811570 | 112.84 | 5.35 | 0.047 | 0.004 |
| FOS | chr14: 75745480-75748937 | 9.81 | 33.22 | 3.386 | 0.005 |
| WBSCR17 | chr7: 70597788-71178584 | 0.15 | 2.13 | 14.538 | 0.007 |
| PXDN | chr2: 1635658-1748291 | 0.42 | 2.62 | 6.177 | 0.009 |
| CD69 | chr12: 9905081-9913497 | 3.67 | 20.3 | 5.53 | 0.01 |
| CLCA1 | chr1: 86934525-86965974 | 2.42 | 25.44 | 10.499 | 0.011 |
| OSM | chr22: 30658818-30662829 | 2.31 | 13.18 | 5.696 | 0.011 |
| CCL5 | chr17: 34198495-34207377 | 55.16 | 18.19 | 0.33 | 0.012 |
| GPR128 | chr3: 100328432-100414323 | 1.81 | 0.01 | 0.005 | 0.012 |
| HLA-DQA1 | chr6: 32605182-32611429 | 110.78 | 32.61 | 0.294 | 0.015 |
| TPSAB1 | chr16: 1290677-1292555 | 17.8 | 104.78 | 5.885 | 0.016 |
| CXCR1 | chr2: 219027567-219031716 | 2.21 | 10.93 | 4.935 | 0.02 |
| CSF2RB | chr22: 37309674-37336479 | 2.62 | 9.28 | 3.539 | 0.021 |
| MUC5B | chr11: 1244294-1283406 | 2.03 | 0.54 | 0.266 | 0.022 |
| AKAP12 | chr6: 151561133-151679694 | 1.42 | 5.11 | 3.605 | 0.023 |
| IL1B | chr2: 113587336-113594356 | 11.15 | 49.25 | 4.416 | 0.026 |
| SAMSN1 | chr21: 15857548-15955723 | 3.7 | 13.69 | 3.697 | 0.026 |
| SLC2A3 | chr12: 8071823-8088892 | 2.92 | 9.11 | 3.119 | 0.027 |
| KRT16 | chr17: 39766030-39769079 | 1.79 | 9.2 | 5.139 | 0.03 |
| MS4A2 | chr11: 59856136-59865940 | 0.35 | 2.72 | 7.762 | 0.03 |
| PRSS33 | chr16: 2833953-2836708 | 0.03 | 1.01 | 28.994 | 0.032 |
| GPR97 | chr16: 57702156-57723290 | 0.96 | 4.57 | 4.77 | 0.032 |
| SOCS3 | chr17: 76352858-76356158 | 8.5 | 25.86 | 3.043 | 0.033 |
| TPSB2 | chr16: 1278335-1280185 | 16.63 | 69.55 | 4.183 | 0.034 |
| FFAR3 | chr19: 35849487-35851389 | 0.13 | 2.65 | 20.367 | 0.034 |
| G0S2 | chr1: 209848669-209849735 | 11.79 | 40.09 | 3.401 | 0.035 |
| TREML2 | chr6: 41157551-41168925 | 0.34 | 1.98 | 5.84 | 0.035 |
| CXCL9 | chr4: 76922622-76928641 | 21.8 | 5.39 | 0.247 | 0.035 |
| PTHLH | chr12: 28111016-28124916 | 1.64 | 17.46 | 10.663 | 0.036 |
| RMRP | chr9: 35657747-35658015 | 128.94 | 18.99 | 0.147 | 0.036 |
| SRGN | chr10: 70847827-70864567 | 43.71 | 133.91 | 3.064 | 0.038 |
| FFAR2 | chr19: 35940616-35942669 | 2.69 | 10.65 | 3.956 | 0.039 |
| EGR1 | chr5: 137801180-137805004 | 4.08 | 10.9 | 2.671 | 0.04 |
| THBS1 | chr15: 39873279-39889668 | 0.99 | 3.31 | 3.352 | 0.042 |
| RGS2 | chr1: 192778168-192781407 | 14.62 | 36.36 | 2.488 | 0.048 |
| LAG3 | chr12: 6881669-6887621 | 5.21 | 1.19 | 0.227 | 0.049 |
| SLC6A8 | chrX: 152953751-152962048 | 25.79 | 66.89 | 2.594 | 0.051 |
| NDRG1 | chr8: 134249413-134309547 | 23.28 | 57.7 | 2.479 | 0.051 |
| CXCR2 | chr2: 218990012-219001976 | 2.67 | 10.97 | 4.103 | 0.052 |
| KRT6A | chr12: 52880957-52887181 | 23.2 | 71.76 | 3.093 | 0.054 |
| DPYSL3 | chr5: 146770370-146889619 | 4.29 | 10.51 | 2.448 | 0.056 |
| DPP4 | chr2: 162848754-162931052 | 1.68 | 5.25 | 3.127 | 0.056 |
| MMP12 | chr13: 102733463-102745764 | 1.46 | 7.71 | 5.283 | 0.058 |
| HCAR3 | chr12: 123199302-123201439 | 7.14 | 24.95 | 3.493 | 0.058 |
| DUOXA2 | chr15: 45406522-45422057 | 52.3 | 19.24 | 0.368 | 0.059 |
| C1QA | chr1: 22963117-22966175 | 35.62 | 13.51 | 0.379 | 0.059 |
| CCL26 | chr7: 75398841-75419064 | 5.68 | 31.76 | 5.589 | 0.06 |
| LGALS7B | chr19: 39279849-39282394 | 49.25 | 123.57 | 2.509 | 0.06 |

### Example 3

### This Example Demonstrates that Targeted RNA-Seq of the Nasal Airway Epithelium Reveals a Th2 Pattern Associated with Atopic Asthma

Targeted RNA-seq (Ampliseq) technology was used to quantitate expression of 105 genes (Table IV). The Ampliseq assay included three gene groups: (1) the top 50 differentially expressed nasal genes in asthma were targeted for confirmation of the whole transcriptome sequencing; (2) the top 20 over- and 10 under-expressed genes in the asthmatic bronchial epithelium according to the study by Woodruff et al (Woodruff P G, et al. Genome-wide profiling identifies epithelial cell genes associated with asthma and with treatment response to corticosteroids. Proc Natl Acad Sci USA 2007; 104: 15858-15863.), to validate the ability of nasal airway expression to proxy bronchial airway expression biomarkers of asthma; (3) to provide genetic and biological context, a select set of 29 “asthma candidate genes,” were targeted defined as such by being either implicated in Genome-wide Association Studies (GWAS) of asthma, other asthma genetic studies, or implicated in mechanistic studies of asthma.

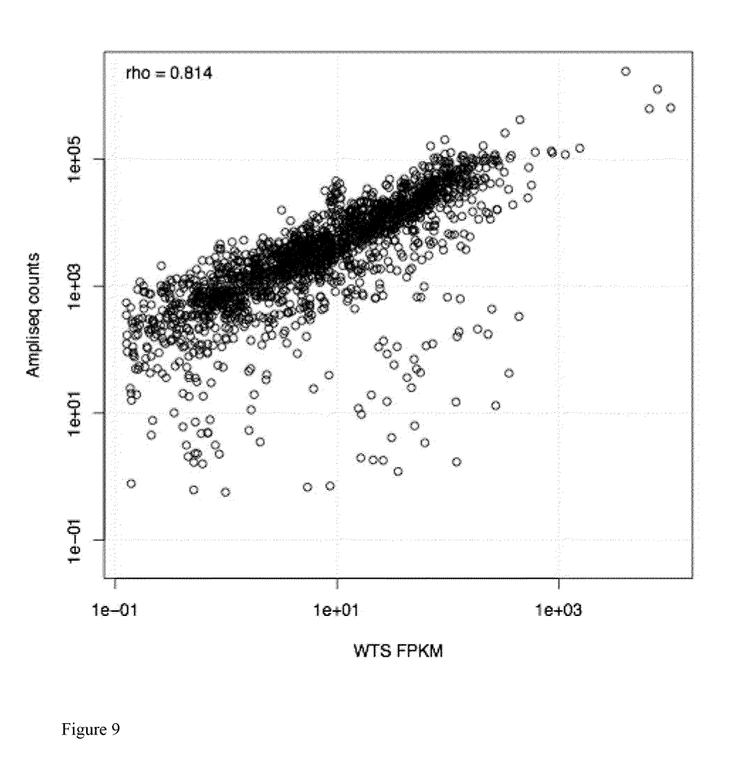

The Ampliseq assay was performed in a larger group of Puerto Rican children with asthma (n=50) and controls (n=50) (Table 1). To validate both gene expression methods, the 20 whole transcriptome subjects were a subset of these 100 subjects (FIG. 9). These subjects were characterized by high rates of atopy in both cases (75.5%) and controls (60%). Additionally, 62% of subjects with asthma and 14% of non-asthmatics self-reported rhinitis. The heterogeneity among both subjects with and without asthma in terms of these allergic phenotypes allowed for the investigation of their relationship with nasal gene expression.

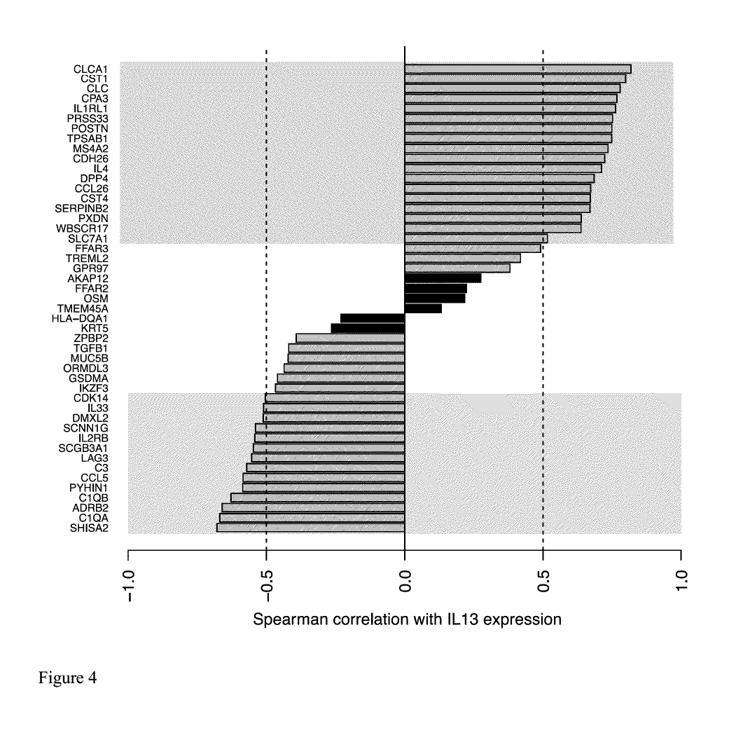

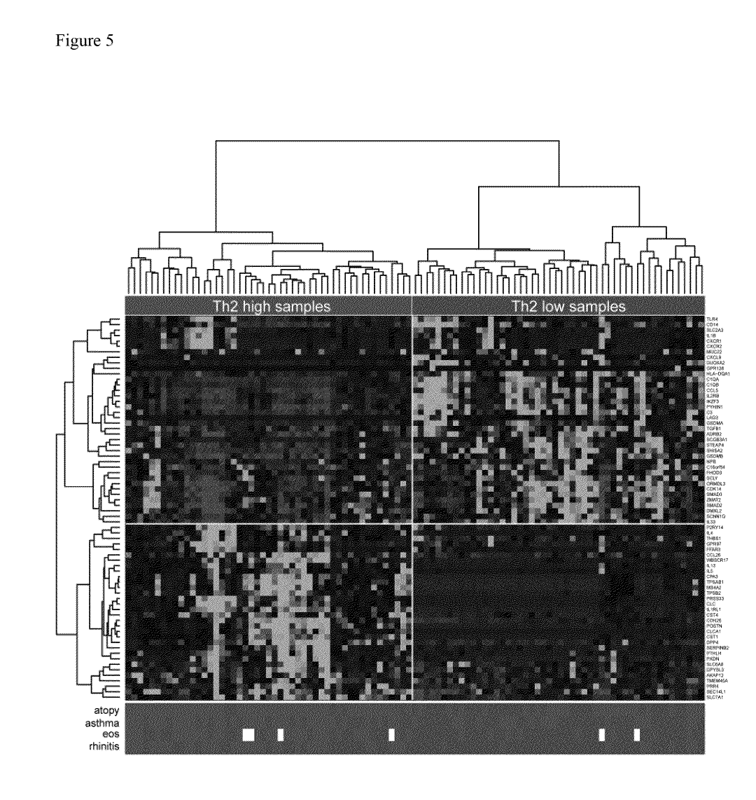

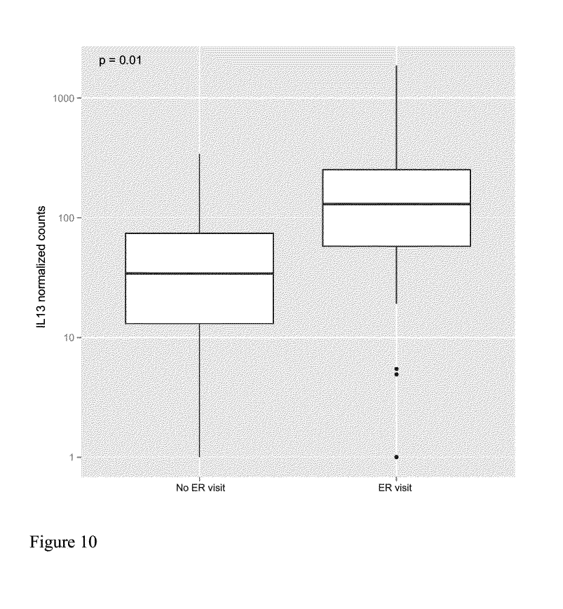

At a sequencing depth of 1.3×10⁶ reads/sample, the inventors were able to detect expression in the nasal airway for 103 of the 105 genes screened. The Ampliseq assay had the sensitivity to measure rare cytokine transcripts such as IL13, IL4, and IL5, which have been below detection level in prior microarray-based studies of bronchial airway gene expression (Woodruff P G, et al. Genome-wide profiling identifies epithelial cell genes associated with asthma and with treatment response to corticosteroids. Proc. Natl Acad Sci USA 2007; 104: 15858-15863.). 48 of the 105 genes assayed were differentially expressed in asthma after correction for multiple testing, including 26 over- and 22 under-expressed genes (Table IV). 17 genes or 57% of the bronchial differential expression set screened, were also significantly differentially expressed in the nasal airways, strongly supporting that gene expression among children with asthma is altered similarly in the bronchial and nasal airways (Table V). Among the differentially expressed genes were epithelial genes (e.g. POSTN, CLCA1, SERPINB2, DPP4, CST1, CST4) and mast cell genes (e.g. TPSAB1, MSA42, CPA3). The cardinal Th2 cytokine, IL13, was upregulated in children with asthma. IL-13 was shown in-vitro to drive upregulation of several genes differentially expressed in the bronchial epithelium of subjects with asthma. Therefore, the ability to measure IL13 transcripts in the airway to examine the correlation between IL13 transcription levels and the other 47 differentially expressed genes in-situ was used. 18 of the 26 upregulated genes exhibited a strong positive correlation (ρ>0.5) with IL13 levels, while 14 of the 22 downregulated genes exhibited a strong negative correlation (ρ<−0.5) with IL13 levels (FIG. 4). Moreover, examining asthma severity, nasal IL13 levels were 3.9-fold higher (p=0.01) in subjects who experienced an asthma exacerbation in the past year versus those who did not (FIG. 10). These data support that differential expression at both epithelial sites (nasal and bronchial) is orchestrated in common by an underlying Th2/IL-13 skew in the systemic immune system.

Genes were then tested for differential expression by atopic status, a measure of systemic Th2 immune system skew. 70 of the 103 expressed genes were differentially expressed by atopic status. Hierarchical clustering of these 70 genes was used in all subjects to determine gene relationships and examine clustering of subjects with varying asthma and allergic status. The first branching point separated Th2-high (IL13 high) from Th2-low (IL13 low) subjects (FIG. 5). The odds ratio for atopy, regardless of asthma status, was 10.33 among subjects with a Th2-high expression pattern (p=3.5×10⁻⁶). A 9.1-fold increased odds for high blood eosinophils among subjects with a Th2-high pattern, another measure of systemic Th2 skew (p=2.6×10⁻⁶). Self-reported rhinitis also clustered tightly with the Th2-high pattern (O.R.=8.3, p=4.1×10⁻⁶). Overall, 42 of the 48 differentially expressed genes in asthma were among the 70 differentially expressed genes in atopy suggesting that the atopic asthmatic subgroup was driving the majority of the differentially expressed genes by asthma status. The odds ratio for atopic asthma among subjects with a Th2-high pattern was 32.6 compared to non-atopic healthy controls (p=6.9×10⁻⁷).

TABLE IV
Samseq differential expression for genes in the targeted nasal Ampliseq analysis

| | | Fold- | | | | |
|---|---|---|---|---|---|---|
| | | Change | | Fold- | | |
| | | in | Asthma | Change | Atopy | Candidate |
| Gene | Source* | Asthmatics | Q value | in Atopy | Q value | Reason |
| ADRB2 | Asthma | 0.89 | 0.034 | 0.706 | 0 | Genetics |
| AKAP12 | WTS | 1.229 | 0.048 | 1.277 | 0 | |
| BCL2A1 | WTS | 1.575 | 0.137 | 0.658 | 0.092 | |
| C16orf54 | Bronch | 0.871 | 0.241 | 0.655 | 0 | |
| C1QA | WTS | 0.606 | 0 | 0.486 | 0 | |
| C1QB | WTS | 0.564 | 0 | 0.436 | 0 | |
| C3 | Bronch | 0.534 | 0.034 | 0.475 | 0.007 | |
| CCL26 | WTS | 1.585 | 0 | 1.922 | 0 | |
| CCL5 | WTS | 0.569 | 0 | 0.636 | 0 | |
| CD14 | Asthma | 0.946 | 0.148 | 0.657 | 0 | Genetics |
| CD69 | WTS | 1.105 | 0.137 | 1.097 | 0.236 | |
| CDH26 | Bronch | 1.553 | 0 | 2.28 | 0 | |
| CDK14 | Bronch | 0.705 | 0 | 0.721 | 0.007 | |
| CLC | WTS | 6.116 | 0 | 33.342 | 0 | |
| CLCA1 | Bronch, WTS | 85.818 | 0 | 579.677 | 0 | |
| CPA3 | Bronch, WTS | 2.755 | 0 | 31.045 | 0 | |
| CSF2RB | WTS | 1.28 | 0.101 | 0.891 | 0.221 | |
| CST1 | Bronch | 23.664 | 0 | 619.824 | 0 | |
| CST4 | Bronch | 2.346 | 0 | 4.42 | 0 | |
| CXCL9 | WTS | 0.661 | 0.118 | 0.595 | 0.007 | |
| CXCR1 | WTS | 0.816 | 0.369 | 0.247 | 0.012 | |
| CXCR2 | WTS | 0.814 | 0.364 | 0.374 | 0.007 | |
| DHX35 | Bronch | 1.021 | 0.369 | 1.039 | 0.306 | |
| DMXL2 | Bronch | 0.943 | 0.046 | 0.938 | 0.017 | |
| DPP4 | WTS | 2.1 | 0 | 2.651 | 0 | |
| DPYSL3 | WTS | 1.006 | 0.339 | 1.388 | 0 | |
| DUOXA2 | WTS | 0.889 | 0.158 | 0.302 | 0 | |
| EGR1 | WTS | 1.159 | 0.272 | 0.871 | 0.258 | |
| FFAR2 | WTS | 2.052 | 0.048 | 0.709 | 0.306 | |
| FFAR3 | WTS | 2.752 | 0 | 2.51 | 0.022 | |
| FHOD3 | Bronch | 0.865 | 0.227 | 0.755 | 0.017 | |
| FOS | WTS | 1.224 | 0.137 | 0.942 | 0.19 | |
| G0S2 | WTS | 0.966 | 0.213 | 0.614 | 0.076 | |
| GPR128 | WTS | 0.479 | 0.158 | 0.24 | 0.012 | |
| GPR97 | WTS | 1.27 | 0.048 | 1.502 | 0.036 | |
| GSDMA | Asthma | 0.523 | 0.034 | 0.45 | 0 | Genetics |
| GSDMB | Asthma | 0.993 | 0.369 | 0.816 | 0.007 | Genetics |
| HCAR3 | WTS | 1 | 0.369 | 1 | 0.325 | |
| HLA- | WTS | 0.318 | 0.046 | 0.362 | 0 | |
| IKZF3 | Asthma | 0.796 | 0.014 | 0.669 | 0 | Genetics |
| IL13 | Asthma | 3.581 | 0.014 | 19.729 | 0 | Bio./Genetics |
| IL18R1 | Asthma | 0.917 | 0.294 | 1.046 | 0.136 | Genetics |
| IL1B | WTS | 1.36 | 0.28 | 0.445 | 0.027 | |
| IL1RL1 | Asthma | 3.59 | 0 | 6.406 | 0 | Genetics |
| IL2RB | Asthma | 0.588 | 0 | 0.574 | 0 | Genetics |
| IL33 | Asthma | 0.792 | 0 | 0.796 | 0 | Genetics |
| IL4 | Asthma | 8.675 | 0.048 | 8.58E+08 | 0 | Bio. |
| IL5 | Asthma | 2.495 | 0.13 | 9.497 | 0 | Bio. |
| KCNIP4 | Asthma | 1 | 0.06 | 1 | 0.306 | Genetics |
| KLK3 | Asthma | 1 | 0.369 | 1 | 0.325 | Genetics |
| KRT14 | Asthma | 1.195 | 0.272 | 0.953 | 0.306 | Bio. |
| KRT16 | WTS | 1.605 | 0.08 | 1.339 | 0.135 | |
| KRT5 | Asthma | 0.683 | 0 | 1.079 | 0.092 | Bio. |
| KRT6A | WTS | 1.425 | 0.178 | 1.18 | 0.314 | |
| LAG3 | WTS | 0.637 | 0 | 0.67 | 0.007 | |
| LGALS7B | WTS | 0.697 | 0.193 | 1.324 | 0.147 | |
| MFGE8 | Asthma | 1.126 | 0.329 | 1.028 | 0.216 | Bio. |
| MMP12 | WTS | 1.385 | 0.054 | 1.217 | 0.236 | |
| MS4A2 | WTS | 3.122 | 0 | 21.388 | 0 | |
| MUC21 | Asthma | 1.159 | 0.272 | 0.856 | 0.092 | Genetics |
| MUC22 | Asthma | 1.05 | 0.329 | 0.756 | 0.007 | Genetics |
| MUC5B | Bronch, WTS | 0.287 | 0 | 0.716 | 0.1 | |
| MUC7 | Asthma | 0.614 | 0.066 | 0.776 | 0.183 | Genetics |
| MXRA7 | Bronch | 0.889 | 0.305 | 1.08 | 0.2 | |
| NDRG1 | WTS | 0.914 | 0.339 | 1.148 | 0.136 | |
| NPB | Bronch | 0.954 | 0.148 | 0.608 | 0.012 | |
| ORMDL3 | Asthma | 0.839 | 0.034 | 0.849 | 0.007 | Genetics |
| OSM | WTS | 3.133 | 0.032 | 0.905 | 0.306 | |
| P2RY14 | Bronch | 1.159 | 0.089 | 1.39 | 0 | |
| POSTN | Bronch | 1.853 | 0.048 | 11.237 | 0 | |
| PRR4 | Bronch | 1.149 | 0.28 | 1.705 | 0 | |
| PRSS33 | WTS | 4.859 | 0 | 38.281 | 0 | |
| PTHLH | WTS | 1.481 | 0.089 | 2.815 | 0 | |
| PXDN | WTS | 1.691 | 0.032 | 2.609 | 0 | |
| PYHIN1 | Asthma | 0.668 | 0 | 0.472 | 0 | Genetics |
| RGS2 | WTS | 1.01 | 0.28 | 0.886 | 0.092 | |
| SAMSN1 | WTS | 1.184 | 0.089 | 1.168 | 0.204 | |
| SCGB3A1 | Bronch | 0.56 | 0.027 | 0.468 | 0 | |
| SCLY | Bronch | 0.828 | 0.066 | 0.811 | 0.007 | |
| SCNN1G | Bronch | 0.68 | 0 | 0.796 | 0.007 | |
| SDK2 | Asthma | 0.899 | 0.148 | 1.079 | 0.202 | Genetics |
| SEC14L1 | Bronch | 0.997 | 0.329 | 1.198 | 0 | |
| SERPINB2 | Bronch | 1.491 | 0 | 1.871 | 0 | |
| SHISA2 | Bronch | 0.684 | 0.034 | 0.559 | 0.007 | |
| SLC2A3 | WTS | 1.098 | 0.213 | 0.728 | 0.027 | |
| SLC6A8 | WTS | 1.13 | 0.095 | 1.306 | 0 | |
| SLC7A1 | Bronch | 1.201 | 0 | 1.365 | 0 | |
| SMAD2 | Asthma | 0.868 | 0.148 | 0.831 | 0 | Genetics |
| SMAD3 | Asthma | 0.969 | 0.294 | 0.923 | 0.007 | Genetics |
| SOCS3 | WTS | 1.167 | 0.339 | 1.023 | 0.183 | |
| SOX2 | Bronch | 0.998 | 0.339 | 1.037 | 0.282 | |
| SRGN | WTS | 1.415 | 0.106 | 0.945 | 0.216 | |
| STEAP4 | Bronch | 0.801 | 0.06 | 0.75 | 0 | |
| STOM | Bronch | 1.05 | 0.213 | 1.041 | 0.325 | |
| TGFB1 | Asthma | 0.836 | 0 | 0.801 | 0 | Bio. |
| THBS1 | WTS | 0.96 | 0.369 | 1.544 | 0.036 | |
| TLR4 | Asthma | 1.117 | 0.339 | 0.612 | 0 | Genetics |
| TMEM45A | Bronch | 1.439 | 0 | 1.484 | 0 | |
| TPSAB1 | Bronch, WTS | 2.522 | 0 | 21.858 | 0 | |
| TPSB2 | WTS | 1.378 | 0.08 | 486599925 | 0 | |
| TREML2 | WTS | 1.814 | 0.026 | 1.177 | 0.135 | |
| TSLP | Asthma | 0.872 | 0.073 | 1.082 | 0.287 | Genetics |
| WBSCR17 | WTS | 2.302 | 0 | 4.497 | 0 | |
| ZMAT2 | Bronch | 1.011 | 0.369 | 0.912 | 0.007 | |
| ZPBP2 | Asthma | 0.073 | 0 | 0.725 | 0.258 | Genetics |

*Reason for inclusion: Bronchial asthma biomarker (Bronch), in top 50 differentially expressed genes from nasal whole transcriptome sequencing data (WTS), and asthma candidate genes (Asthma), subclassified as genetic or bioiogical (Bio) candidates.
ADRB2 (Lima J nJ, et al. Clin Pharmacol Ther 1999; 65: 519-525.; Martinez F D, et al. J Clin Invest 1997; 100: 3184-3188.; Silverman E K, et al. J Allergy Clin Immunol 2003; 112: 870-876.)
CD14 (Zambelli-Weiner A, et al. J Allergy Clin Immunol 2005; 115: 1203-1209.)
GSDMA, GSDMB, IKZF3, IL33, (Moffatt M F, et al. N Engl J Med 2010; 363: 1211-1221.; Torgerson D G, et al. Nat Genet 2011; 43: 887-892.)
IL2RB (Moffatt M F, et al. N Engl J Med 2010; 363: 1211-1221)
IL13 (Moffatt M F, et al. N Engl J Med 2010; 363: 1211-1221.; Li X, et al. J Allergy Clin Immunol 2010; 125: 328-335 e311.; Wills-Karp M, et al. Science 1998; 282: 2258-2261.)
IL18R1, IL1RL1 (Moffatt M F, et al. N Engl J Med 2010; 363: 1211-1221.; Torgerson D G, et al. Nat Genet 2011; 43: 887-892.; Gudbjartsson D F, et al. Nat Genet 2009; 41: 342-347.)
IL4 (Rankin J A, et al. Proc Natl Acad Sci USA 1996; 93: 7821-7825.)
IL5 (Foster P S, et al. J Exp Med 1996; 183: 195-201.)
KCNIP4 (Himes B E, et al. PLoS One 2013; 8: e56179.)
KLK3 (Myers R A, et al. J Allergy Clin Immunol 2012; 130: 1294-1301.)
KRT14, KRT5 (Kicic A, et al. Am J Respir Crit Care Med 2006; 174: 1110-1118.)
MFGE8 (Kudo M, et al. Proc Natl Acad Sci USA 2013; 110: 660-665.)
MUC21, MUC22 (Galanter J M, et al. The GALA II Study. J Allergy Clin Immunol 2013; In Press.)
MUC7, SDK2 (Torgerson D G, et al. J Allergy Clin Immunol 2012; 130: 76-82 e12.)
ORMDL3, PYHIN1, SMAD3, ZPBP2 (Moffatt M F, et al. N Engl J Med 2010; 363: 1231-1221.; Torgerson D G, et al. Nat Genet 2011; 43: 887-892.)
SMAD2 (Gignoux C R, et al. American Society of Human Genetics Conference Abstract 2012.)
TGFB1 (Scherf W, et al. Eur J Immunol 2005; 35: 198-206.)
TLR4 (Yang I A, et al. Genes Immun 2004; 5: 41-45.)
TSLP (Hirota T, et al. Nat Genet 2011; 43: 893-896; Torgerson D G, et al. Nat Genet 2011; 43: 887-892)

### Example 4

### This Example Demonstrates Asthma-Specific and Th2-Independent Gene Expression in the Nasal Transcriptome

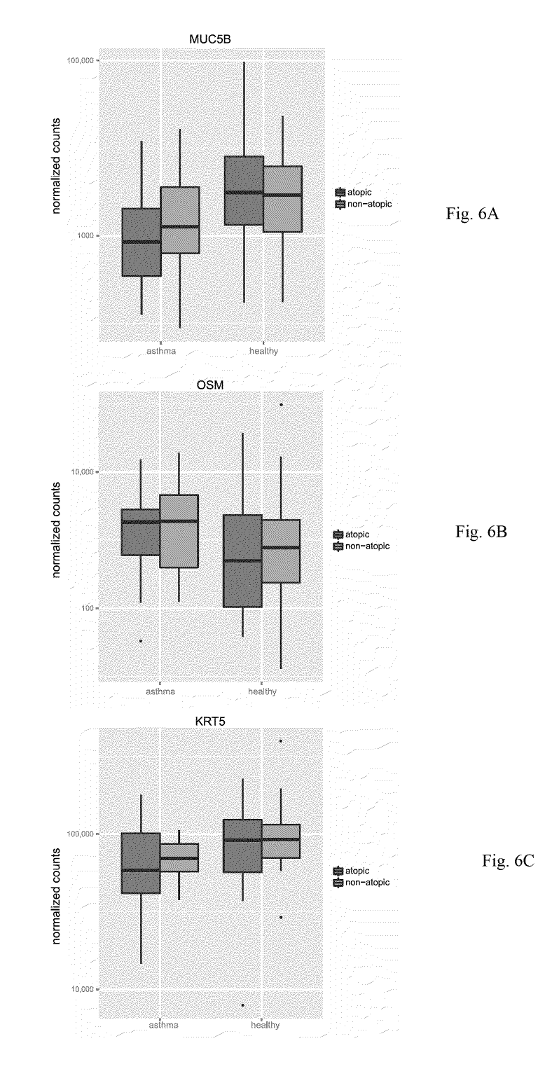

In contrast, six genes (cytokeratin-5 (KRT5), mucin 5b (MUC5B), zona pellucida binding protein 2 (ZPBP2), triggering receptor expressed on myeloid cells-like 2 (TREML2), oncostatin M (OSM), free fatty acid receptor 2 (FFAR2)) were associated with asthma and not atopy. None of these six genes were strongly correlated with IL-13 levels (0.5>r>−0.5) (FIG. 4). Differential expression in these genes was driven by both atopic and non-atopic subjects with asthma (FIG. 6).

### Example 5

### This Example Demonstrates Asthma GWAS Genes Differentially Expressed in Nasal Airway Epithelium

Detection of gene expression in the nasal airway brushings of all 16 asthma GWAS genes screened was determined with the Ampliseq assay. Nasal expression of 9 (56.3%) and 11 (68.8%) of these candidate genes were found associated with asthma and atopy, respectively. The genes and their fold-changes in expression are listed in Table V. Among the differentially expressed genes verified through large GWAS meta-analyses were the cytokine IL-33 and its receptor, IL1RL1 (ST2). Expression of IL1RL1 was strongly upregulated in both atopy and asthma, whereas IL-33 was downregulated in both of these groups.

The 17q21 asthma GWAS risk locus contains 5 genes. In the nasal Ampliseq assay ORMDL3 and GSDMB were found to be highly expressed, IKZF3 moderately expressed, and GSDMA and ZPBP2 were lowly expressed. ORMDL3, IKZF3, and GSDMA were all downregulated in both atopic and asthmatics subjects. ZPBP2 and GSDMB were downregulated in just asthmatic and atopic subjects, respectively.

TABLE V
Bronchial biomarkers found to be differentially expressed in the nasal
airways of subjects with asthma by Ampliseq assay

| | Log2 fold-change | Log2 fold-change |
|---|---|---|
| Gene | nasal* | bronchial^{†} |
| POSTN | 0.89 | 2.071 |
| CPA3 | 1.462 | 1.817 |
| TPSAB1 | 1.335 | 1.064 |
| SERPINB2 | 0.576 | 1.835 |
| CLCA1 | 6.423 | 2.632 |
| CST4 | 1.23 | 1.245 |
| CST1 | 4.565 | 2.176 |
| SLC7A1 | 0.264 | 0.21 |
| CDH26 | 0.635 | 0.477 |
| TMEM45A | 0.525 | −0.923 |
| SCNN1G | −0.556 | −0.925 |
| DMXL2 | −0.085 | −0.31 |
| MUC5B | −1.801 | −0.863 |
| SCGB3A1 | −0.837 | −1.091 |
| CDK14 | −0.504 | −0.235 |
| C3 | −0.905 | −1.001 |
| SHISA2 | −0.548 | −1.07 |

*Values derived from samseq analysis of Ampliseq data
^{†}From Woodruff, et al microarray data

### Example 6

This example demonstrates that DPP4 is an asthma/atopy biomarker strongly induced in the airway epithelial by TL-13 stimulation. DPP4 expression is proinflammatory in airway epithelial cells as judged by IL-8 gene expression. DPP4 overexpression inhibits human rhinovirus-16 (HRV-16) infection of airway epithelial cells, possibly through induction of a type 3 interferon response. DPP4 may play an important role in lung Th2 inflammation and have protective effects against HRV infection.

DPP4 expression in children (controls and asthmatics) with varying atopy status was determined. In addition, the DPP4 effects on inflammation and HRV-16 infection in both nasal and bronchial airway epithelial cells was determined.

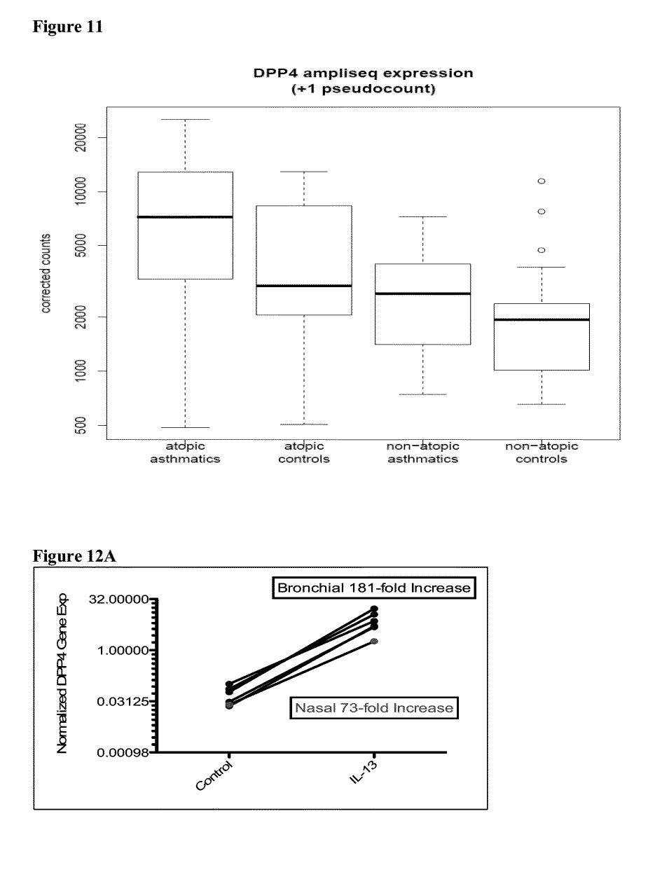

FIG. 11 shows that the nasal epithelium DPP4 gene expression is unregulated in both atopy and asthma. The DPP4 gene expression levels are higher in atopy and asthma compared to healthy control subject levels.

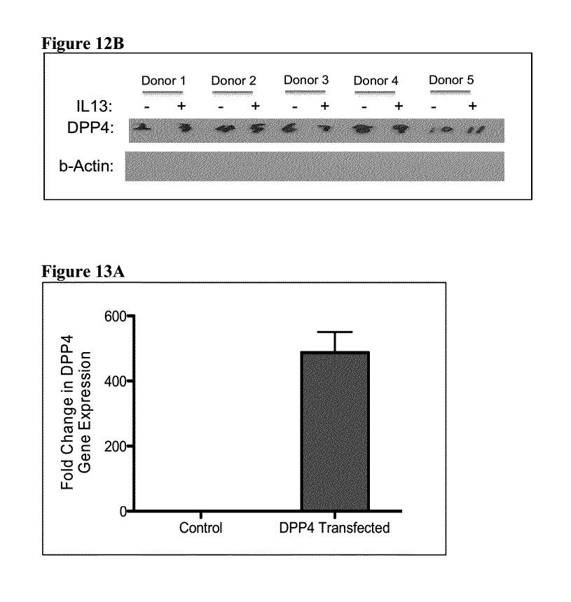

FIGS. 12A and 12 B show that DPP4 gene and protein expression in airway epithelial cells is strongly induced by IL-13 treatment.

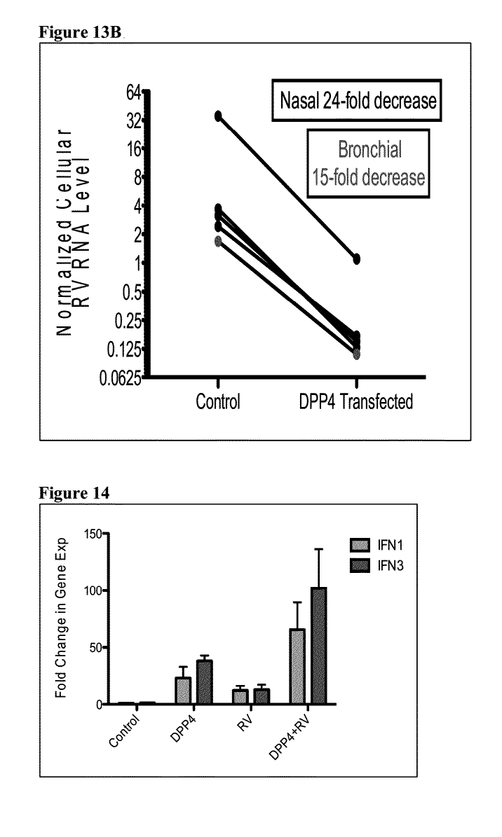

FIGS. 13A and 13B show that DPP4 over-expression inhibits HRV-16 infection of airway epithelial cells.

FIG. 14 shows that DPP4 overexpression induces a strong interferon response.

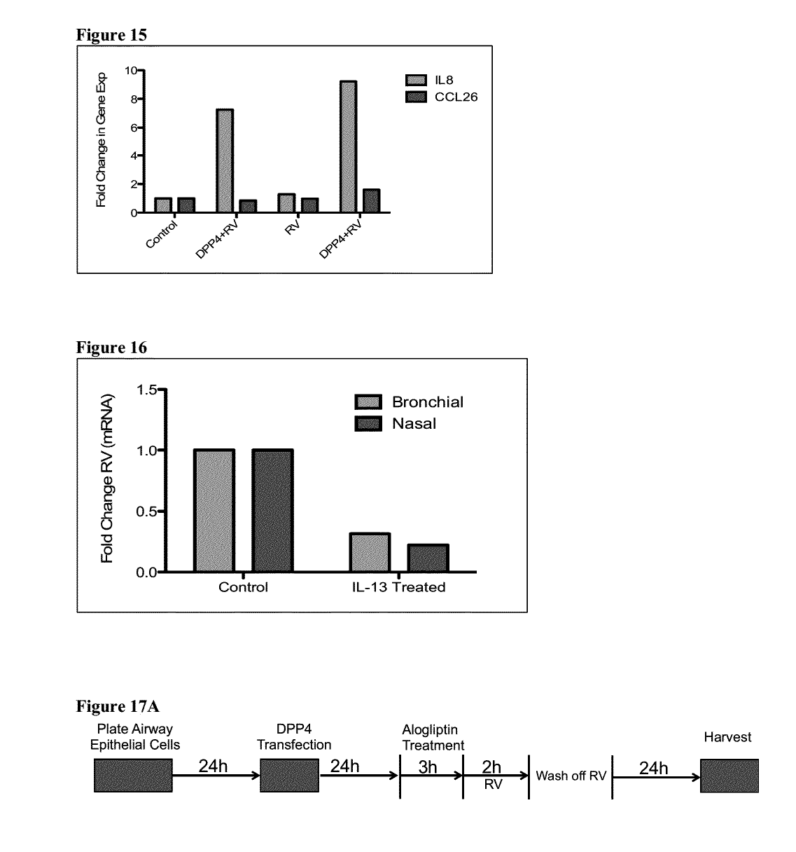

FIG. 15 shows that DPP4 overexpression induces IL8 but not CCL26 expression

FIG. 16 shows that IL-13 treatment decreases HRV-16 infection of ALI differentiated airway epithelial cells.

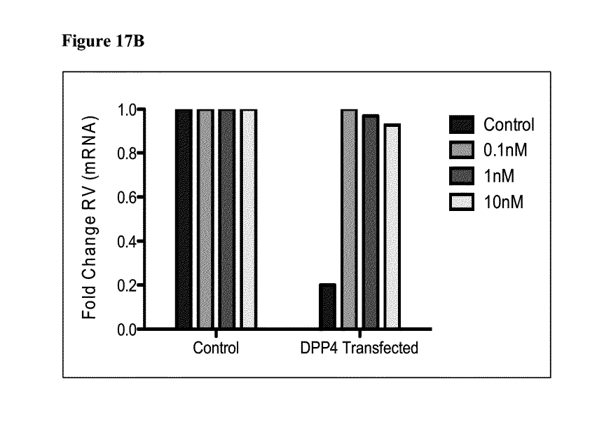

FIGS. 17A and 17B show that a DPP4 inhibitor reverses DPP4 downregulation of bronchial epithelial cells (BEC) HRV-16 Infection.

While various embodiments of the present invention have been described in detail, it is apparent that modifications and adaptations of those embodiments will occur to those skilled in the art. It is to be expressly understood, however, that such modifications and adaptations are within the scope of the present invention.

### REFERENCES

1. Natlonal Health Interview Survey 2004-2011. In: Prevention CfDCa, editor. Natlonal Center for Health Statistics.
2. Woodruff P G, Boushey H A, Dolganov G M, Barker C S, Yang Y H, Donnelly S, et al. Genome-wide profiling identifies epithelial cell genes associated with asthma and with treatment response to corticosteroids. Proc Natl Acad Sci USA 2007; 104: 15858-15863.
3. Woodruff P G, Modrek B, Choy D F, Jia G, Abbas A R, Ellwanger A, et al. T-helper type 2-driven inflammation defines major subphenotypes of asthma. Am J Respir Crit Care Med 2009; 180: 388-395.
4. Corren J, Lemanske R F, Flanania N A, Korenblat P E, Parsey M V, Arron J R, et al. Lebrikizumab treatment in adults with asthma. N Engl J Med 2011; 365: 1088-1098.
5. Wenzel S, Ford L. Pearlman D, Spector S, Sher L, Skobieranda F, et al. Dupilumab in persistent asthma with elevated eosinophil levels. N Engl J Med 2013; 368: 2455-2466.
6. Harkema J R, Carey S A, Wagner J G. The nose revisited; a brief review of the comparative structure, function, and toxicologic pathology of the nasal epithelium. Toxicol Pathol 2006; 34: 252-269.
7. Sridhar S, Schembri F, Zeskind J, Shah V, Gustafson A M, Steiling K, et al. Smoking-induced gene expression changes in the bronchial airway are reflected in nasal and buccal epithelium. BMC Genomics 2008; 9: 259.
8. Guajardo J R, Schleifer K W, Daines M O, Ruddy R I M, Aronow B J, Wills-Karp M, et al. Altered gene expression profiles in nasal respiratory epithelium reflect stable versus acute childhood asthma. J Allergy Clin Immunol 2005; 115: 243-251.
9. Borrell L N, Nguyen E A, Roth L A, Oh S S, Tcheurekdjian H, Sen S, et al. Childhood obesity and asthma control in the GALA II and SAGE II studies. Am J Respir Crit Care Med 2013; 187: 697-702.
10. Kumar R, Nguyen E A, Roth L A, Oh S S, Gignoux C R, Huntsman S, et al. Factors associated with degree of atopy in Latino children in a nationwide pediatric sample: The Genes environments and Admixture in Latino Asthmatics (GALA II) study. J Allergy Clin Immunol 2013.
11. Nishimura K K, Galanter J M, Roth L A, Oh S S, Thakur N, Nguyen E A, et al. Early Life Air Pollution and Asthma Risk in Minority Children: The GALA II & SAGER Studies. Am J Respir Crit Care Med 2013.
12. Szefler S J, Wenzel S, Brown R, Erzurum S C, Fahy J V, Hamilton R G, et al. Asthma outcomes: biomarkers. J Allergy Clin Immunol 2012; 129: S9-23.
13. Kim D, Pertea G, Trapnell C, Pinientel H, Kelley R, Salzberg S L. TopHat2; accurate alignment of transcriptomes in the presence of insertions, deletions and gene fusions. Genome Biol 2013; 14: R36.
14. Trapnell C, Roberts A, Goff L, Pertea G, Kim D, Kelley D R, et al. Differential gene and transcript expression analysis of RNA-seq experiments with TopHat and Cufflinks. Nat Protoc 2012; 7: 562-578.
15. Trapnell C, Williams B A, Pertea G, Mortazavi A, Kwan G, van Baren M J, et al. Transcript assembly and quantification by RNA-Seq reveals unannotated transcripts and isoform switching during cell differentiation. Nat Biotechnol 2010; 28: 511-515.
16. Goff L, Trapnell C, Kelley D. CummeRbund: Visualization and Exploration of Cufflinks High-throughput Sequencing Data. 2012.
17. Li J, Tibshirani R. Finding consistent patterns: A nonparametric approach for identifying differential expression in RNA-Seq data. Stat Methods Med Res 2011.
18. Beane J, Vick J, Schembri F, Anderlind C, Gower A, Campbell J, et al. Characterizing the impact of smoking and lung cancer on the airway transcriptome using RNA-Seq. Cancer Prev Res (Phila) 2011; 4: 803-817.
19. Hackett N R, Butler M W, Shaykhiev R, Salit J, Omberg L, Rodriguez-Flores J L, et al. RNA-Seq quantification of the human small airway epithelium transcriptome. BMC Genomics 2012.; 13: 82.
20. Ramskold D, Wang E T, Burge C B, Sandberg R. An abundance of ubiquitously expressed genes revealed by tissue transcriptome sequence data. PLoS Comput Biol 2009; 5: e1000598.
21. Bhakta N R, Solberg O D, Nguyen C P, Nguyen C N, Arron J R, Fahy J V, et al. A qPCR-based metric of Th2 airway inflammation in asthma. Clin Transl Allergy 2013; 3: 24.
22. Yurtsever Z. Sala-Rahanal M, Randolph D T, Scheaffer S M, Roswit W T, Alevy Y G, et al. Self-cleavage of human CLCA1 protein by a novel internal metalloprotease domain controls calcium-activated chloride channel activation. J Biol Chem 2012; 287: 42138-42149.
23. McDougall C M, Blaylock M G, Douglas J G, Brooker R J, Helms P J, Walsh G M. Nasal epithelial cells as surrogates for bronchial epithelial cells in airway inflammation studies. Am J Respir Cell Mol Biol 2008; 39: 560-568.
24. Kabesch M, Schedel M, Carr D, Woitsch B, Fritzsch C, Weiland S K, et al. IL-4/IL-13 pathway genetics strongly influence serum EgE levels and childhood asthma. J Allergy Clin Immunol 2006; 117: 269-274.
25. Simpson J L, Baines K J, Boyle M J, Scott R J, Gibson P G. Oncostatin M (OSM) is increased in asthma with incompletely reversible airflow obstruction. Exp Lung Res 2009; 35: 781-794.
26. Moffatt M F, Gut I G, Demenais F, Strachan D P, Bouzigon E, Heath S, et al. A large-scale, consortium-based genomewide association study of asthma. N Engl J Med 2010; 363: 1211-1221.
27. Torgerson D G, Ampleford E J, Chiu G Y, Gauderman W J, Gignoux C R, Graves P E, et al. Meta-analysis of genome-wide association studies of asthma in ethnically diverse North American populations. Nat Genet 2011; 43: 887-892.
28. Li X, Howard T D, Zheng S L, Haselkom T, Peters S P, Meyers D A, et al. Genome-wide association study of asthma identifies RAD50-IL13 and HLA-DR/DQ regions. J Allergy Clin Immunol 2010; 125: 328-335 e311.
29. Gudbjartsson D F, Bjornsdottir U S, Halapi E, Helgadottir A, Sulem P, Jonsdottir G M, et al. Sequence variants affecting eosinophil numbers associate with asthma and myocardial infarction. Nat Genet 2009; 41: 342-347.
30. Himes B E, Sheppard K, Berndt A, Leme A S, Myers R A, Gignoux C R, et al. Integration of mouse and human genome-wide association data identifies KCNIP4 as an asthma gene. PLoS One 2013; 8: e56179.
31. Myers R A, Himes B E, Gignoux C R, Yang J J, Gauderman W J, Rebordosa C, et al. Further replication studies of the EVE Consortium meta-analysis identifies 2 asthma risk loci in European Americans. J Allergy Clin Immunol 2012; 130: 1294-1301.
32. Gignoux C R, Torgerson D G, Galanter J, Roth I, Eng C, Hu D, et al. Admixture Mapping From Existing Genome-Wide Association Data Identifies SMAD2 As A Population-Specific Risk Factor For Asthma In Latinos. ATS Conference Abstract 2013.
33. Hirota T, Takahashi A, Kubo M, Tsunoda T, Tomita K, Doi S. et al. Genome-wide association study identifies three new susceptibility loci for adult asthma in the Japanese population. Nat Genet 2011; 43: 893-896.
34. Hannon G J. FASTX-Toolkit. http://hannonlabcshledu/fastx_toolkit/indexhtml.
35. Kim D, Pertea G, Trapnell C, Pimentel H. Kelley R, Salzberg S L. TopHat2: accurate alignment of transcriptomes in the presence of insertions, deletions and gene fusions. Genome Biol 2013; 14: R36.
36. Trapnell C, Pachter L, Salzberg S L. TopHat: discovering splice junctions with RNA-Seq. Bioinformatics 2009; 25: 1105-1111.
37. Langmead B, Salzberg S L. Fast gapped-read alignment with Bowtie 2. Nat Methods 2012; 9: 357-359,
38. Trapnell C, Williams B A, Pertea G, Mortazavi A, Kwan G, van Baren M J, et al. Transcript assembly and quantification by RNA-Seq reveals unannotated transcripts and isoform switching during cell differentiation. Nat Biotechnol 2010; 28: 511-515.
39. Trapnell C, Roberts A, Goff L, Pertea G, Kim D, Kelley D R, et al. Differential gene and transcript expression analysis of RNA-seq experiments with TopHat and Cufflinks. Nat Protoc 2012; 7: 562-578.
40. Goff L T, C. Kelley, D. cummeRbund: Analysis, exploration, manipulation, and visualization of Cufflinks high-throughput sequencing data. wwwbioconductororg/packages/release/bioc/html/cummeRbundhtml 2012.
41. Woodruff P G, Boushey H A, Dolganov G M, Barker C S, Yang Y H, Donnelly S, et at Genome-wide profiling identifies epithelial cell genes associated with asthma and with treatment response to corticosteroids. Proc Natl Acad Sci USA 2007; 104: 15858-15863.
42. Wheeler D L, Barrett T, Benson D A, Bryant S H, Canese K, Chetvernin V, et al. Database resources of the Natlonal Center for Biotechnology information. Nucleic Acids Res 2008; 36: D13-21.
43. Beane J, Vick J, Schembri F, Anderlind C, Gower A, Campbell J, et al. Characterizing the impact of smoking and lung cancer on the airway transcriptome using RNA-Seq. Cancer Prev Res (Phila) 2011; 4: 803-817.
44. Hackett N R, Butler M W, Shaykhiev R, Salit J, Omberg L, Rodriguez-Flores J L, et al. RNA-Seq quantification of the human small airway epithelium transcriptome. BMC Genomics 2012; 13: 82.
45. Ramskold D, Wang E T, Burge C B, Sandberg R. An abundance of ubiquitously expressed genes revealed by tissue transcriptome sequence data. PLoS Comput Biol 2009; 5: e1000598.
46. Irizarry R A, Warren D, Spencer F, Kim I F, Biswal S, Frank B C, et al. Multiple-laboratory comparison of microarray platforms. Nat Methods 2005; 2: 345-350.
47. Li J, Tibshirani R. Finding consistent patterns: A nonparametric approach for identifying differential expression in RNA-Seq data. Stat Methods Med Res 2011.
48. Anders S, Huber W. Differential expression analysis for sequence count data. Genome Biol 2010; 11: R106.
49. Breiman L. Random Forests. Machine Learning 2001: 5-32.
50. Liaw A W, M. C. Classification and Regression by randomForest. R News 2002: 18-22.
51. Lima J J, Thomason D B, Mohamed M H, Eberle L V, Self T H, Johnson J A. Impact of genetic polymorphisms of the beta2-adrenergic receptor on albuterol bronchodilator pharmacodynamics. Clin Pharmacol Ther 1999; 65: 519-525.
52. Martinez F D, Graves P E, Baldini M, Solomon S, Erickson R. Association between genetic polymorphisms of the beta2-adrenoceptor and response to albuterol in children with and without a history of wheezing. J Clin Invest 1997; 100: 3184-3188.
53. Silverman E K, Kwiatkowski D J, Sylvia J S, Lazarus R, Drazen J M, Lange C, et al. Family-based association analysis of beta2-adrenergic receptor polymorphisms in the childhood asthma management program. J Allergy Clin Immunol 2003; 112: 870-876.
54. Zambelli-Weiner A, Ehrlich E, Stockton M L, Grant A V, Zhang S, Levett P N, et al. Evaluation of the CD14/-260 polymorphism and house dust endotoxin exposure in the Barbados Asthma Genetics Study. J Allergy Clin Immunol 2005; 115: 1203-1209.
55. Moffatt M F, Gut I G, Demenais F, Strachan D P, Bouzigon E, Heath S. et al. A large-scale, consortium-based genomewide association study of asthma. N Engl J Med 2010; 363: 1211-1221.
56. Torgerson D G, Ampleford E J, Chiu G Y, Gauderman W J, Gignoux C R, Graves P E, et al. Meta-analysis of genome-wide association studies of asthma in ethnically diverse North American populations. Nat Genet 2011; 43: 887-892.
57. Li X, Howard T D, Zheng S L, Haselkorn T, Peters S P, Meyers D A, et al. Genome-wide association study of asthma identifies RAD50-IL13 and HLA-DR/DQ regions. J Allergy Clin Immunol 2010; 125: 328-335 e311.
58. Wills-Karp M, Luyimbazi J, Xu X, Schofield B, Neben T Y, Karp C L, et al. Interleukin-13: central mediator of allergic asthma. Science 1998; 282: 2258-2261.
59. Gudbjartsson D F, Bjornsdottir U S, Halapi E, Helgadottir A, Sulem P, Jonsdottir G M, et al. Sequence variants affecting eosinophil numbers associate with asthma and myocardial infarction. Nat Genet 2009; 41: 342-347.
60. Rankin J A, Picarella D E, Geba G P, Temann U A, Prasad B, DiCosmo B, et al, Phenotypic and physiologic characterization of transgenic mice expressing interleukin 4 in the lung: lymphocytic and eosinophilic inflammation without airway hyperreactivity. Proc Natl Acad. Sci USA 1996; 93: 7821-7825.
61. Foster P S, Hogan S P, Ramsay A J, Matthaei K I, Young I G. Interleukin 5 deficiency abolishes eosinophilia, airways hyperreactivity, and lung damage in a mouse asthma model. J Exp Med 1996; 183: 195-201.
62. Himes B E, Sheppard K, Berndt A, Leme A S, Myers R A, Gignoux C R, et al. Integration of mouse and human genome-wide association data identifies KCNIP4 as an asthma gene. PLoS One 2013; 8: e56179.
63. Myers R A, Himes B E, Gignoux C R, Yang J J, Gauderman W J, Rebordosa C, et al. Further replication studies of the EVE Consortium meta-analysis identifies 2 asthma risk loci in European Americans. J Allergy Clin Immunol 2012; 130: 1294-1301.
64. Kicic A, Sutanto E N, Stevens P T, Knight D A, Stick S M. Intrinsic biochemical and functional differences in bronchial epithelial cells of children with asthma. Am J Respir Crit Care Med 2006; 174: 1110-1118.
65. Kudo M, Khalifeh Soltani S M, Sakuma S A, McKleroy W, Lee T H, Woodruff P G, et al. Mfge8 suppresses airway hyperresponsiveness in asthma by regulating smooth muscle contraction. Proc Natl Acad Sci USA 2013; 110: 660-665.
66. Galanter J M, Gignoux C R, Torgerson D G, Roth L A, Eng C, Oh S S, et al. GWAS and admixture mapping identify different asthma-associated loci in Latinos: The GALA II Study. J Allergy Clin Immunol 2013; In Press.
67. Torgerson D G, Gignoux C R, Galanter J M, Drake K A, Roth L A, Eng C, et al. Case-control admixture mapping in Latino populations enriches for known asthma-associated genes. J Allergy Clin Immunol 2012; 130: 76-82 e12,
68. Gignoux C R, Torgerson D G, Galanter J M, Roth L A, Eng C, Hu D, et al. Admixture Mapping From Existing Genome-Wide Association Data Identifies SMAD2 As A Population-Specific Risk Factor For Asthma In Latinos. American Society of Human Genetics Conference Abstract 2012.
69. Scherf W, Burdach S, Hansen G. Reduced expression of transforming growth factor beta 1 exacerbates pathology in an experimental asthma model. Eur J Immunol 2005; 35: 198-206.
70. Yang I A, Barton S J, Rorke S, Cakebread J A, Keith T P, Clough J B, et al. Toll-like receptor 4 polymorphism and severity of atopy in asthmatics. Genes Immun 2004; 5: 41-45,
71. Hirota T, Takahashi A, Kubo M, Tsunoda T, Tomita K, Doi S, et al. Genome-wide association study identifies three new susceptibility loci for adult asthma in the Japanese population. Nat Genet 2011; 43: 893-896.

What is claimed is:

1. A method of identifying a subject at risk of exacerbation of a respiratory disease comprising:
a. obtaining a nasal epithelium sample from the subject;
b. determining the expression level of any one or more genes that had been determined to be strongly correlated with IL-13 expression from the nasal epithelium sample from the subject;
c. comparing the expression level from the subject in step (I)) to a control level; and
d. identifying the subject as being at risk of exacerbation of a respiratory disease if an altered gene expression level of the one or more genes from the subject in step (b) as compared to the control level in step (c) is determined.

2. The method of claim 1, wherein the respiratory disease is asthma.

3. The method of claim 1, wherein the nasal epithelium sample is obtained by a method selected from the group consisting of nasal brushing or swabbing, nasal lavage, scrapings from nasal mucosa and blown secretions.

4. The method of claim 1, wherein the expression level of the one or more genes that had been determined to be strongly correlated with IL-13 expression is determined by Next-generation based sequencing and transcript quantification.

5. The method of claim 1, wherein the one or more genes that had been determined to be strongly correlated with IL-13 expression is selected from the group consisting of IL-13, IL-4, IL-5, DPP4, ADRB2, AKAP12, BCL2A1, C16orf54, C1QA, C1QB, C3, CCL26, CCL5, CD14, CD69, CDH26, CDK14, CLC, CLCA1, CPA3, CST2RB, CST1, CST4, CXCL9, CXCR1, CXCR2, DHX35, DMXL2, DPYSL3, DUOXA2, EGR1, FFAR2, FFAR3, FHOD3, FOS, G0S2, GPR128, GPR97, GSDMA, GSDMB, HCAR3, HLA-DQA1, IKZF3, IL18R1, IL1B, IL1RL1, IL2RB, IL33, KCNIP4, KLK3, KRT14, KRT16, KRT5, KRT6A, LAG3, LGALS7B, MFGE8, MMP12, MS4A2, MUC21, MUC22, MUC5B, MUC7, MXRA7, NDRG1, NPB, ORMDL3, OSM, P2RY14, POSTN, PRR4, PRSS33, PTHLH, PXDN, PYHIN1, RGS2, SAMSN1, SCGB3A1, SCLY, SCNN1G, SDK2, SEC14L1, SERPINB2, SHISA2, SLC2A3, SLC6A8, SLC7A1, SMAD2, SMAD3, SOCS3, SOX2, SRGN, STEAP4, STOM, TGFB1, THBS1, TLR4, TMEM45A, TPSAB1, TPSB2, TREML2, TSLP, WBSCR17, ZMAT2, and ZPBP2 and combinations thereof.

6. The method of claim 1, wherein the gene expression level of the any one or more of the genes from the subject in step (b) as compared to the control level in step (c) is altered if the expression level of the one or more genes is over-expressed or under-expressed as compared to the control level.

7. A method of identifying a subject having a respiratory disease who is responsive to treatment with an inhibitor selected from the group consisting of an IL-13, IL-4, IL-5 and Th2 pathway inhibitor comprising:
a. obtaining a nasal epithelium sample from the subject,
b. determining the expression level of any one or more genes that had been determined to be strongly correlated with IL-13 expression from the nasal epithelium sample from the subject;
c. comparing the expression level from the subject in step (b) to a control level; and
d. identifying the subject as being responsive to treatment with the inhibitor if an altered gene expression level of the any one or more of the genes from the subject in step (b) as compared to the control level in step (c) is determined.

8. The method of claim 7, wherein the respiratory disease is asthma.

9. The method of claim 7, wherein the nasal epithelium sample is obtained by a method selected from the group consisting of nasal brushing or swabbing, nasal lavage, scrapings from nasal mucosa and blown secretions.

10. The method of claim 7, wherein the expression level of the one or more genes that had been determined to be strongly correlated with IL-13 expression is determined by Next-generation based sequencing and transcript quantification.

11. The method of claim 7, wherein the one or more genes that had been determined to be strongly correlated with IL-13 expression is selected from the group consisting of IL-13, IL-4, IL-5, DPP4, ADRB2, AKAP12, BCL2A1, C16orf54, C1QA, C1QB, C3, CCL26, CCL5, CD14, CD69, CDH26, CDK14, CLC, CLCA1, CPA3, CSF2RB, CST1, CST4, CXCL9, CXCR1, CXCR2, DHX35, DMXL2, DPYSL3, DUOXA2, EGR1, FFAR2, FFAR3, FHOD3, FOS, G0S2, GPR128, GPR97, GSDMA, GSDMB, HCAR3, HLA-DQA1, IKZF3, IL18R1, IL1B, IL1RL1, IL2RB, IL33, KCNIP4, KLK3, KRT14, KRT16, KRT5, KRT6A, LAG3, LGALS7B, MFGE8, MMP12, MS4A2, MUC21, MUC22, MUC5B, MUC7, MXRA7, NDRG1, NPB, ORMDL3, OSM, P2RY14, POSTN, PRR4, PRSS33, PTHLH, PXDN, PYHIN1, RGS2, SAMSN1, SCGB3A1, SCLY, SCNN1G SDK2, SEC1413, SERPINB2, SHISA2, SLC2A3, SLC6A8, SLC7A1, SMAD2, SMAD3, SOCS3, SOX2, SRGN, STEAP4, STOM, TGFB1, THBS1, TLR4, TMEM45A, TPSAB1, TPSB2, TREML2, TSLP, WBSCR17, ZMAT2, and ZPBP2 and combinations thereof.

12. The method of claim 7, wherein the gene expression level of the any one or more of the genes from the subject in step (b) as compared to the control level in step (c) is altered if the expression level of the one or more genes is over-expressed or under-expressed as compared to the control level.

13. The method of claim 7, wherein the subject identified is administered a therapeutically effective amount of an inhibitor selected from the group consisting of an IL-13, IL-4, IL-5 and Th2 pathway inhibitor.

14. A method of identifying subject having a Type 2 helper T cell-high (Th2-high) asthma subphenotype comprising:
a. obtaining a nasal epithelium sample from the subject;
b. determining the expression level of one or more genes that had been determined to be strongly correlated with IL-13 expression in the nasal epithelium sample from the subject,
c. comparing the expression level from the subject in step (b) to a control level; and
d. identifying the subject as having the Th2-high asthma subphenotype if an altered gene expression level of the any one or more of the genes from the subject in step (b) as compared to the control level in step (c) is determined.

15. The method of claim 14, wherein the nasal epithelium sample is obtained by a method selected from the group consisting of nasal brushing or swabbing, nasal lavage, scrapings from nasal mucosa and blown secretions.

16. The method of claim 14, wherein the expression level of the one or more genes that had been determined to be strongly correlated with IL-13 expression is determined by Next-generation based sequencing and transcript quantification.

17. The method of claim 14, wherein the one or more genes that had been determined to be strongly correlated with IL-13 expression is selected from the group consisting of IL-13, IL-4, IL-5, DPP4, ADRB2, AKAP12, BCL2A1, C16orf54, C1QA, C1QB, C3, CCL26, CCL5, CD14, CD69, CDH26, CDK14, CLC, CLCA1, CPA3, CSF2RB, CST1, CST4, CXCL9, CXCR1, CXCR2, DHX35, DMXL2, DPYSL3, DUOXA2, EGR1, FFAR2, FFAR3, FHOD3, FOS, G0S2, GPR128, GPR97, GSDMA, GSDMB, HCAR3, HLA-DQA1, IKZF3, IL18R1, IL1B, IL1RL1, IL2RB, IL33, KCNIP4, KLK3, KRT14, KRT16, KRT5, KRT6A, LAG3, LGALS7B, MFGE8, MMP12, MS4A2, MUC21, MUC22, MUC5B, MUC7, MXRA7, NDRG1, NPB, ORMDL3, OSM, P2RY14, POSTN, PRR4, PRSS33, PTHLH, PXDN, PYHIN1, RGS2, SAMSN1, SCGB3A1, SCLY, SCNN1G, SDK2, SEC14L1, SERPINB2, SHISA2, SLC2A3, SLC6A8, SLC7A1, SMAD2, SMAD3, SOCS3, SOX2, SRGN, STEAP4, STOM, TGFB1, THBS1, TLR4, TMEM45A, TPSAB1, TPSB2, TREML2, TSLP, WBSCR17, ZMAT2, and ZPBP2 and combinations thereof.

18. The method of claim 14, wherein the gene expression level of the any one or more of the genes from the subject in step (b) as compared to the control level in step (c) is altered if the expression level of the one or more genes is over-expressed or under-expressed as compared to the control level.

19. A method of identifying a subject having an inflammatory disease resistant to corticosteroid treatment comprising
a. obtaining a nasal epithelium sample from the subject;
b. determining the expression level of one or more genes that had been determined to be strongly correlated with IL-13 expression in the nasal epithelium sample from the subject,
c. comparing the expression level from the subject in step (b) to a control level; and
d. identifying the subject as having an inflammatory disease resistant to corticosteroid treatment if an altered gene expression level of the any one or more of the genes from the subject in step (b) as compared to the control level in step (c) is determined.

20. The method of claim 19, wherein the inflammatory disease is asthma.

21. The method of claim 19, wherein the nasal epithelium sample is obtained by a method selected from the group consisting of nasal brushing or swabbing, nasal lavage, scrapings from nasal mucosa and blown secretions.

22. The method of claim 19, wherein the expression level of the one or more genes that had been determined to be strongly correlated with IL-13 expression is determined by Next-generation based sequencing and transcript quantification.

23. The method of claim 19, wherein the one or more genes that had been determined to be strongly correlated with IL-13 expression is selected from the group consisting of IL-13, IL-4, IL-5, DPP4, ADRB2, AKAP12, BCL2A1, C16orf54, C1QA, C1QB, C3, CCL26, CCL5, CD14, CD69, CDH26, CDK14, CLC, CLCA1, CPA3, CSF2RB, CST1, CST4, CXCL9, CXCR1, CXCR2, DHX35, DMXL2, DPYSL3, DUOXA2, EGR1, FFAR2, FFAR3, FHOD3, FOS, G0S2, GPR128, GPR97, GSDMA, GSDMB, HCAR3, HLA-DQA1, IKZF3, IL18R1, IL1B, IL1RL1, IL2RB, IL33, KCNIP4, KLK3, KRT14, KRT16, KRT5 KRT6A, L4G3, LGALS7B, MFGE8, MMP12, MS4A2, MUC21, MUC22, MUC5B, MUC7, MXRA7, NDRG1, NPB, ORMDL3, OSM, P2RY14, POSTN, PRR4, PRSS33, PTHLH, PXDN, PYHIN1, RGS2, SAMSN1, SCGB3A1, SCLY, SCNN1G, SDK2, SEC14L1, SERPINB2, SHISA2, SLC2A3, SLC6A8, SLC7A1, SMAD2, SMAD3, SOCS3, SOX2, SRGN, STEAP4, STOM, TGFB1, THBS1, TLR4, TMEM45A, TPSAB1 TPSB2, TREML2, TSLP, WBSCR17, ZMAT2, and ZPBP2 and combinations thereof.

24. The method of claim 19, wherein the gene expression level of the any one or more of the genes from the subject in step (b) as compared to the control level in step (c) is altered if the expression level of the one or more genes is over-expressed or under-expressed as compared to the control level.
